(19) United States
(12) Patent Application Publication
(10) Pub. No.: US 2012/0108563 A1
(43) Pub. Date: May 3, 2012

(54) Methods Of Treating Liposarcoma

(75) Inventor: David S. Grayzel, Winchester, MA (US)

(73) Assignee: Infinity Pharmaceuticals, Inc., Cambridge, MA (US)

(21) Appl. No.: 13/321,036

(22) PCT Filed: May 19, 2010
(86) PCT No.: PCT/US10/35403

§ 371 Date: Jan. 3, 2012

Related U.S. Application Data
(60) Provisional application No. 61/179,555, filed on May 19, 2009.

(51) Int. Cl. A61K 31/395; A61P 35/00

(52) U.S. Cl. 514/183

(57) ABSTRACT

Provided herein are methods of treating liposarcoma in a subject, the method comprising administering to the subject a therapeutically effective amount of an Hsp90 inhibitor.

### RELATED APPLICATION

This application claims the benefit of priority to U.S. Provisional Patent Application Ser. No. 61/179,555, filed May 19, 2009; the contents of which are hereby incorporated by reference.

### BACKGROUND

Liposarcoma is malignancy arising out of the adipose tissue and is one of the most common forms of soft tissue sarcoma (STS), comprising about 10% to 30% of STS cases. Roughly 2000 new cases of liposarcoma are diagnosed each year, and nearly 700 deaths from the disease occur annually.

### SUMMARY

Provided herein are methods of treating liposarcoma in a subject, the method comprising administering to the subject a therapeutically effective amount of an Hsp90 inhibitor. Exemplary Hsp90 inhibitors useful for practicing the present invention are described in detail herein.

Details of the invention are set forth in the accompanying Description and Exemplification as described herein. Other features, objects, and advantages of the invention will be apparent from this description and from the claims.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Certain compounds of the present invention can comprise one or more asymmetric centers, and thus can exist in various isomeric forms, e.g., enantiomers and/or diastereomers. The compounds provided herein may be in the form of an individual enantiomer, diastereomer or geometric isomer, or may be in the form of a mixture of stereoisomers, including racemic mixtures and mixtures enriched in one or more stereoisomer. In certain embodiments, the compounds of the invention are enantiopure compounds. In certain other embodiments, mixtures of stereoisomers are provided.

Furthermore, certain compounds, as described herein may have one or more double bonds that can exist as either the cis or trans, or the E or Z isomer, unless otherwise indicated. The invention additionally encompasses the compounds as individual isomers substantially free of other isomers, and alternatively, as mixtures of various isomers, e.g., racemic mixtures of E/Z isomers or mixtures enriched in one E/Z isomer.

Where a particular enantiomer is preferred, it may be provided substantially free of the corresponding enantiomer, i.e., optically enriched. “Optically enriched,” as used herein, means that the compound is made up of a greater proportion of one enantiomer compared to the other. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from mixtures by methods known to those skilled in the art, including chiral high pressure liquid chromatography (HPLC) and the formation and crystallization of chiral salts; or preferred enantiomers may be prepared by asymmetric syntheses. See, for example, Wilen et al., Tetrahedron (1977) 33:2725; Enantiomers, Racemates and Resolutions (Jacques, Ed., Wiley Interscience, New York, 1981); Stereochemistry of Carbon Compounds (E. L. Eliel, Ed., McGraw-Hill, NY, 1962); and Tables of Resolving Agents and Optical Resolutions p. 268 (E. L. Eliel, Ed., Univ. of Notre Dame Press, Notre Dame, Ind. 1972).

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, alone or as part of another group, “alkyl” refers to a monoradical of a straight-chain or branched saturated hydrocarbon group having from 1 to 10 carbon atoms (“C₁₋₁₀ alkyl”). In some embodiments, an alkyl group has 1 to 8 carbon atoms (“C₁₋₈ alkyl”). In some embodiments, an alkyl group has 1 to 6 carbon atoms (“C₁₋₆ alkyl”). In some embodiments, an alkyl group has 1 to 4 carbon atoms (“C₁₋₄ alkyl”). In some embodiments, an alkyl group has 2 to 6 carbon atoms (“C₂ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈), n-nonyl (C₉) n-decyl (C₁₀), and the like. Unless otherwise specified, each instance of an alkyl group is independently unsubstituted (an “unsubstituted alkyl”) or substituted (a “substituted alkyl”) with 1-5 substituents as described herein. In certain embodiments, the alkyl group is an unsubstituted C₁₋₁₀ alkyl. In certain embodiments, the alkyl group is a substituted C₁₋₁₀ alkyl.

The terms “alkoxyl” refers to an alkyl group, as defined herein, having an oxygen radical attached thereto. In one embodiment, alkoxyl groups include methoxy, ethoxy, propyloxy, tert-butoxy and the like. The alkyl portion of an alkoxy group is sized like the alkyl groups, and can be substituted by the same groups that are suitable as substituents on alkyl groups, to the extent permitted by the available valences.

As used herein, alone or as part of another group, “alkenyl” refers to a monoradical of a straight-chain or branched hydrocarbon group having from 2 to 10 carbon atoms and one or more carbon-carbon double bonds (“C₂₋₁₀ alkenyl”). In some embodiments, an alkenyl group has 2 to 10 carbon atoms (“C₂₋₁₀ alkenyl”). In some embodiments, an alkenyl group has 2 to 8 carbon atoms (“C₂₋₈ alkenyl”). In some embodiments, an alkenyl group has 2 to 6 carbon atoms (“C₂₋₆ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄) and the like. Examples of C₂₋₆ alkenyl groups include the aforementioned C₂ alkenyl groups as well as pentenyl (C₅), pentadienyl (C₅), hexenyl (C₆) and the like. Additional examples of alkenyl include heptenyl (C₇), octenyl (C₈), octatrienyl (C₈) and the like. Unless otherwise specified, each instance of an alkenyl group is independently unsubstituted (an “unsubstituted alkenyl”) or substituted (a “substituted alkenyl”) with 1-5 substituents as described herein. In certain embodiments, the alkenyl group is an unsubstituted C₂₋₁₀ alkenyl. In certain embodiments, the alkenyl group is a substituted C₂₋₁₀ alkenyl.

As used herein, alone or as part of another group, “alkynyl” refers to a monoradical of a straight-chain or branched hydrocarbon group having from 2 to 10 carbon atoms and one or more carbon-carbon triple bonds (“C₂₋₁₀ alkynyl”). In some embodiments, an alkynyl group has 2 to 8 carbon atoms (“C₂₋₈ alkynyl”). In some embodiments, an alkynyl group has 2 to 6 carbon atoms (“C₂₋₆ alkynyl”). In some embodiments, an alkynyl group has 2 to 4 carbon atoms (“C₂₋₄ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄) and the like. Examples of C₂₋₆ alkenyl groups include the aforementioned C₂₋₄ alkynyl groups as well as pentynyl (C₅), hexynyl (C₆) and the like. Additional examples of alkynyl include heptynyl (C₇), octynyl (C₈) and the like. Unless otherwise specified, each instance of an alkynyl group is independently unsubstituted (an “unsubstituted alkynyl”) or substituted (a “substituted alkynyl”) with 1-5 substituents as described herein. In certain embodiments, the alkynyl group is an unsubstituted C₂₋₁₀ alkynyl. In certain embodiments, the alkynyl group is a substituted C₂₋₁₀ alkynyl.

As used herein, alone or as part of another group, “carbocyclyl” refers to a radical of a non-aromatic cyclic hydrocarbon group having from 3 to 10 ring carbon atoms (“C₃₋₁₀ carbocyclyl”), and no ring heteroatoms. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Examples of C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₈ carbocyclyl groups include the aforementioned C₃₋₆ carbocyclyl groups as well as cycloheptyl (C₇), cycloheptadienyl (C₇), cycloheptatrienyl (C₇), cyclooctyl (C₈), and the like. A carbocyclyl group can be either monocyclic (“monocyclic carbocyclyl”), bicyclic or tricyclic (“bicyclic carbocyclyl” or “tricyclic carbocyclyl”, e.g., containing a fused, bridged or spiro ring system), and can be saturated or can contain one or more carbon-carbon double or triple bonds. “Carbocyclyl” also includes groups in which the carbocyclyl ring is fused to/substituted with one or more aryl groups, as defined herein, such as indane, indene or tetrahydronaphthyl, where the point of attachment is on the carbocyclyl ring. Unless otherwise specified, each instance of a carbocyclyl group is independently unsubstituted (an “unsubstituted carbocyclyl”) or substituted (a “substituted carbocyclyl”) with 1-5 substituents as described herein. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with 1-5 substituents as described herein. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, alone or as part of another group, “heterocycloalkyl” refers to a radical of a cyclic 3- to 14-membered non-aromatic ring system having ring carbon atoms and 1 to 4 ring heteroatoms, each heteroatom independently selected from nitrogen, oxygen and sulfur (“3-14 membered heterocycloalkyl”). In heterocycloalkyl groups that contain one or more nitrogen atoms, the point of attachment can be a carbon or nitrogen atom, as valency permits. A heterocycloalkyl group can either be monocyclic (“monocyclic heterocycloalkyl”), bicyclic or tricyclic (“bicyclic heterocycloalkyl” or “tricyclic heterocycloalkyl”, e.g., containing a fused, bridged or spiro ring system), and can be saturated or can contain one or more carbon-carbon double or triple bonds. In some embodiments, a heterocycloalkyl group is a cyclic 3-12 membered non-aromatic ring system having ring carbon atoms and 1-4 ring heteroatoms, wherein each heteroatom is independently selected from nitrogen, oxygen and sulfur (“3-12 membered”). In some embodiments, a heterocycloalkyl group is a cyclic 5-10 membered non-aromatic ring system having ring carbon atoms and 1-4 ring heteroatoms, wherein each heteroatom is independently selected from nitrogen, oxygen and sulfur (“5-10 membered heterocycloalkyl”). In some embodiments, a heterocycloalkyl group is a cyclic 5-8 membered non-aromatic ring system having ring carbon atoms and 1-4 ring heteroatoms, wherein each heteroatom is independently selected from nitrogen, oxygen and sulfur (“5-8 membered heteroaryl”). In some embodiments, a heterocycloalkyl group is a cyclic 5-6 membered non-aromatic ring system having ring carbon atoms and 1-4 ring heteroatoms, wherein each heteroatom is independently selected from nitrogen, oxygen and sulfur (“5-6 membered heterocycloalkyl”). In some embodiments, 5-6 membered heterocycloalkyl has 1-3 ring heteroatoms selected from nitrogen, oxygen and sulfur. In some embodiments, the 5-6 membered heterocycloalkyl has 1-2 ring heteroatoms selected from nitrogen, oxygen and sulfur. In some embodiments, the 5-6 membered heterocycloalkyl has 1 ring heteroatom selected from nitrogen, oxygen and sulfur. Exemplary 3-membered heterocycloalkyls containing 1 heteroatom include, without limitation, azirdinyl, oxiranyl, thiorenyl. Exemplary 4-membered heterocycloalkyls containing 1 heteroatom include, without limitation, azetidinyl, oxetanyl and thietanyl. Exemplary 5-membered heterocycloalkyls containing 1 heteroatom include, without limitation, tetrahydrofuranyl, dihydrofuranyl, tetrahydrothiophenyl, dihydrothiophenyl, pyrrolidinyl, and dihydropyrrolyl. Exemplary 5-membered heterocycloalkyls containing 2 heteroatoms include, without limitation, dioxolanyl, oxathiolanyl and dithiolanyl. Exemplary 6-membered heterocycloalkyl groups containing 1 heteroatom include, without limitation, piperidinyl, tetrahydropyranyl, dihydropyridinyl, and thianyl. Exemplary 6-membered heterocycloalkyl groups containing 2 heteroatoms include, without limitation, piperazinyl, morpholinyl, dithianyl, dioxanyl. Exemplary 7-membered heterocycloalkyl groups containing 1 heteroatom include, without limitation, azepanyl, oxepanyl and thiepanyl. Exemplary 8-membered heterocycloalkyl groups containing 1 heteroatom include, without limitation, azocanyl, oxecanyl and thiocanyl, heterocycloalkyl groups can be saturated or can contain one or more carbon-carbon double bonds, carbon-nitrogen double bonds, or carbon-carbon triple bonds. “Heterocycloalkyl” also includes groups in which a heterocycloalkyl ring is fused to/substituted with one or more aryl or heteroaryl groups, such as indolinyl, phthalimidyl, naphthalimidyl, chromanyl, tetrahydroquinolinyl or tetrahydroisoquinolinyl, wherein the point of attachment is on the heterocycloalkyl ring. For bicyclic or tricyclic heterocycloalkyl groups wherein one ring does not contain a heteroatom (e.g., decahydroquinoline, decahydroquinoxaline and the like) the point of attachment may be on either ring, i.e., either the ring bearing a heteroatom or the ring that does not contain a heteroatom. Unless otherwise specified, each instance of heterocycloalkyl is independently unsubstituted (an “unsubstituted heterocycloalkyl”) or substituted (a “substituted heterocycloalkyl”) with 1-5 substituents as described herein. In certain embodiments, the heterocycloalkyl group is an unsubstituted 3-14 membered heterocycloalkyl. In certain embodiments, the heterocycloalkyl group is a substituted 3-14 membered heterocycloalkyl.

As used herein, alone or as part of another group, “aryl” refers to a radical of a monocyclic, bicyclic or tricyclic aromatic ring system (e.g., having 6, 10, or 14 π electrons shared in a cyclic array) having 6-14 ring carbon atoms and zero heteroatoms provided in the aromatic ring system (“C₆₋₁₄ aryl”). In certain embodiments, an aryl group has 6 to 10 ring carbon atoms (“C₆₋₁₀ aryl”). In some embodiments, an aryl group has 6 ring carbon atoms (“C₆ aryl”; e.g., phenyl). In some embodiments, an aryl group has 10 ring carbon atoms (“C₁₋₁₀ aryl”; e.g., naphthyl such as 1-naphthyl and 2-naphthyl). In some embodiments, an aryl group has 14 ring carbon atoms (“C₁₋₄ aryl”; e.g., anthracyl). Also included within the scope of the term “aryl”, as it is used herein, is a group in which the aryl ring is fused to/substituted with one or more non-aromatic carbocyclyl or heterocycloalkyl rings, such as indanyl, phthalimidyl, naphthalimidyl, or tetrahydronaphthyl, and the like, where the radical or point of attachment is on the aryl ring. Unless otherwise specified, each instance of an aryl group is independently unsubstituted (an “unsubstituted aryl”) or substituted (a “substituted aryl”) with 1-5 substituents as described herein. In certain embodiments, the aryl group is an unsubstituted C₆₋₁₄ aryl. In certain embodiments, the aryl group is a substituted C₆₋₁₄ aryl.

As used herein, alone or part of another group, “aralkyl” refers to a C₁₋₁₀ alkyl group as defined herein substituted by a C₆₋₁₄ aryl group as defined herein, wherein the point of attachment is on the alkyl group (“C₁₋₁₀ aralkyl”).

As used herein, alone or as part of another group, “heteroaryl” refers to a radical of a 5-14 membered aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. In some embodiments, a heteroaryl group is a 5-12 membered aromatic ring system having ring carbon atoms and 1-4 ring heteroatoms provided in the aromatic ring system, wherein each heteroatom is independently selected from nitrogen, oxygen and sulfur (“5-12 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen and sulfur (“5-6 membered heteroaryl”). In some embodiments, the 5-6 membered heteroaryl has 1-3 ring heteroatoms selected from nitrogen, oxygen and sulfur. In some embodiments, the 5-6 membered heteroaryl has 1-2 ring heteroatoms selected from nitrogen, oxygen and sulfur. In some embodiments, the 5-6 membered heteroaryl has 1 ring heteroatom selected from nitrogen, oxygen and sulfur. Exemplary 5-membered heteroaryls containing 1 heteroatom include, without limitation, pyrrolyl, furanyl and thiophenyl. Exemplary 5-membered heteroaryls containing 2 heteroatoms include, without limitation, imidazolyl, pyrazolyl, oxazolyl, isoxazolyl, thiazolyl, and isothiazolyl. Exemplary 5-membered heteroaryls containing 3 heteroatoms include, without limitation, triazolyl, oxadiazolyl, thiadiazolyl. Exemplary 5-membered heteroaryls containing 4 heteroatoms include, without limitation, tetrazolyl. Exemplary 6-membered heteroaryls containing 1 heteroatom include, without limitation, pyridinyl. Exemplary 6-membered heteroaryls containing 2 heteroatoms include, without limitation, pyridazinyl, pyrimidinyl and pyrazinyl. Exemplary 6-membered heteroaryls containing 3 or 4 heteroatoms include, without limitation, triazinyl and tetrazinyl, respectively. Exemplary 7 membered heteroaryls containing 1 heteroatom include, without limitation, azepinyl, oxepinyl and thiepinyl. Exemplary 5,6-bicyclic heteroaryls include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryls include, without limitation, naphthyridinyl, pteridinyl, quinolinyl, isoquinolinyl, cinnolinyl, quinoxalinyl, phthalazinyl and quinazolinyl. Exemplary tricyclic heteroaryls include, without limitation, phenanthridinyl, dibenzofuranyl, carbazolyl, acridinyl, phenothiazinyl, phenoxazinyl and phenazinyl. Also included within the scope of the term “heteroaryl”, as it is used herein, is a group in which the heteroaryl ring is fused to/substituted with one or more non-aromatic carbocyclyl or heterocycloalkyl as defined herein, e.g., such as indanyl, phthalimidyl, naphthalimidyl, or tetrahydronaphthyl, and the like, where the radical or point of attachment is on the heteroaryl ring. For bicyclic or tricyclic heteroaryl groups wherein one ring does not contain a heteroatom (e.g., indolyl, quinolinyl, carbazolyl and the like) the point of attachment may be on either ring, i.e., either the ring bearing a heteroatom (e.g., 2-indolyl) or the ring that does not contain a heteroatom (e.g., 5-indolyl). Unless otherwise specified, each instance of a heteroaryl group is independently unsubstituted (an “unsubstituted heteroaryl”) or substituted (a “substituted heteroaryl”) with 1-5 substituents as described herein. In certain embodiments, the heteroaryl group is an unsubstituted 5-14 membered heteroaryl. In certain embodiments, the heteroaryl group is a substituted 5-14 membered heteroaryl.

As used herein, alone or part of another group, “heteroaralkyl” refers to a C₁₋₁₀ alkyl group as defined herein substituted by a 5-14 membered heteroaryl group as defined herein, wherein the point of attachment is on the alkyl group (“C₁₋₁₀ heteroaralkyl”).

The term “halide” refers to fluoro (—F), bromo (—Br), iodo (—I) or chloro (—Cl).

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “substituted” refers to a chemical group, such as alkyl, cycloalkyl aryl, and the like, wherein at least one hydrogen is replaced with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocycloalkyl, aromatic or heteroaromatic moieties, —CF₃, —CN, or the like. The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds. In many embodiments, however, any single substituent has fewer than the 100 total atoms. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “pharmaceutically acceptable acid” refers to inorganic or organic acids that exhibit no substantial toxicity. Examples of pharmaceutically acceptable acids include, but are not limited to, hydrochloric acid, hydrobromic acid, hydroiodic acid, sulfuric acid, sulfamic acid, phosphoric acid, nitric acid, p-toluenesulfonic acid, phenylsulfonic acid, methanesulfonic acid, trifluoromethylsulfonic acid, 10-camphorsulfonic acid, naphthalene-1,5-sulfonic acid, cyclamic acid, naphthalene-2-sulfonic acid, thiocyanic acid, fumaric acid, malic acid, maleic acid, succinic acid, benzoic acid, acetic acid, citric acid, tartaric acid, carbonic acid, propionic acid, glycolic acid, stearic acid, lactic acid, tartaric acid, citric acid, ascorbic acid, palmitic acid, hydroxymaleic acid, phenylacetic acid, glutamic acid, salicyclic acid, sulfanilic acid, 2-acetoxybenzoic acid, ethane disulfonic acid, oxalic acid, thiocyanic acid, isothionic acid (2-hydroxyethanesulfonic acid) and the like (see, e.g., Berge et al. “Pharmaceutical Salts”, J. Pharm. Sci. (1977) 66:1-19). Conjugate bases of the above-referenced pharmaceutically acceptable acids correspond to chloride, bromide, iodide, HSO₄⁻, sulfamate, H₂PO₄⁻, nitrate, p-toluenesulfonate, benzenesulfonate, methylsulfonate, trifluoromethylsulfonate,10-camphorsulfonate, naphthalene-1-sulfonic acid-5-sulfonate, cyclamate (i.e., N-cyclohexylsulfamate), naphthalene-2-sulfonate, thiocyanate, fumarate, malate, maleate, succinate, benzoate, acetate, citrate, tartrate, carbonate, propionate, glucolate, stearate, lactate, tartrate, citrate, ascorbate, palmitate, hydroxymaleate, phenylacetate, glutamate, salicylate, sulfanilate, 2-acetoxybenzoate, ethan-1-sulfonic acid-2-sulfonate, thiocyanate, oxalate and isothionate.

The term “pharmaceutically acceptable salt” or “salt” refers to a salt of a compound and a pharmaceutically acceptable acid. Suitable pharmaceutically acceptable salts of compounds include acid addition salts which may, for example, be formed by mixing a solution of the compound with a pharmaceutically acceptable acid.

The term “co-salt” refers to compositions in which a compound (e.g., a compound of formulae I or II) is present or complexed with at least one other compound, such as an amino acid.

### DETAILED DESCRIPTION

Provided herein is a method of treating liposarcoma in a subject, the method comprising administering to the subject a therapeutically effective amount of an Hsp90 inhibitor.

### Liposarcoma

Liposarcoma is a lipogenic tumor of large deep-seated connective tissue spaces. Liposarcomas are most commonly found in the extremities and in the retroperitoneum, and less often in the head and neck area. These tumors are most likely to arise from deep-seated, well-vascularized structures than from submucosal or subcutaneous fat. Liposarcomas of all subtypes can occur in the cutis and the subcutis, but their primary occurrence in the skin is rare. Fusion proteins created by chromosomal abnormalities are key components of mesenchymal cancer development. Abnormalities of bands in the 12q13-15 region have been associated with the development of certain liposarcomas. The most common chromosomal translocation is the FUS-CHOP fusion gene, which encodes a transcription factor necessary for adipocyte differentiation.

Five categories of liposarcomas are generally recognized: (1) well differentiated (also known as atypical lipomatous tumors), which includes lipoma-like (adipocytic), sclerosing, and inflammatory subtypes; (2) dedifferentiated; (3) myxoid (both high and low grade); (4) round cell; and (5) pleomorphic. In some circumstances, tumors can have a combination of these morphologic types; these tumors are classified as combined or mixed-type liposarcomas.

Well-differentiated liposarcomas usually contain a predominance of mature fat cells with relatively few, widely scattered lipoblasts. In the sclerosing subtype of a well-differentiated liposarcoma, collagen fibrils that encircle fat cells and lipoblasts make up a prominent part of the matrix. Well differentiated liposarcomas are tumors of low grade malignancy that may recur locally but generally do not metastasize. Well differentiated liposarcomas can be characterized by a gain of 12q14, including over-expression of MDM2 and/or CDK4.

Dedifferentiated liposarcomas commonly arise from well differentiated liposarcomas. These tumors can rapidly proliferate locally and can metastasize. Dedifferentiated liposarcomas occur most frequently in the retroperitoneum and can be characterized by a gain of 12q14, including over-expression of MDM2 and/or CDK4.

Myxoid liposarcoma, the most common type of liposarcoma, is diagnosed by the observation of a delicate plexiform capillary network that is associated with both primitive mesenchymelike cells and a variable number of lipoblasts. The stroma contains a large proportion of myxoid ground substance (ie, hyaluronic acid), in which numerous microcysts may form. Myxoid LPS and round-cell liposarcomas share a key genetic defect (t12; 16), (q13; p11), which results in fusion of the transcription factor gene CHOP with FUS. Thus, round cell liposarcoma, in which lipoblasts are interspersed among sheets of poorly differentiated round cells, is thought to represent the high-grade counterpart of myxoid liposarcoma. Myxoid and round cell liposarcomas are present most often in the extremities.

Pleomorphic liposarcoma is characterized by a mixture of bizarre, often multivacuolated lipoblasts and atypical stromal cells, many of which contain highly abnormal mitotic figures. Hemorrhagic and necrotic areas are common and lipoblasts are present. Pleomorphic liposarcomas most often occur in the extremities and have a high risk for metastases.

In some embodiments, the Hsp90 inhibitor is used to treat a well differentiated liposarcoma. In some embodiments, the Hsp90 inhibitor is used to treat a well differentiated liposarcoma characterized by over-expression of CDK4. In some embodiments, the Hsp90 inhibitor is used to treat a well differentiated liposarcoma characterized by over-expression of MDM2. In some embodiments, the Hsp90 inhibitor is used to treat a well differentiated liposarcoma characterized by over-expression of both CDK4 and MDM2.

In some embodiments, the Hsp90 inhibitor is used to treat a dedifferentiated liposarcoma. In some embodiments, the Hsp90 inhibitor is used to treat a dedifferentiated liposarcoma characterized by over-expression of CDK4. In some embodiments, the Hsp90 inhibitor is used to treat a dedifferentiated liposarcoma characterized by over-expression of MDM2. In some embodiments, the Hsp90 inhibitor is used to treat a dedifferentiated liposarcoma characterized by over-expression of both CDK4 and MDM2. In some embodiments, the Hsp90 inhibitor is used to treat a dedifferentiated liposarcoma and a well differentiated liposarcoma.

In some embodiments, the Hsp90 inhibitor is used to treat a myxoid liposarcoma. In other embodiments, the Hsp90 inhibitor is used to treat a round cell liposarcoma. In some embodiments, the Hsp90 inhibitor is used to treat a myxoid and a round cell liposarcoma.

In certain embodiments, the Hsp90 inhibitor is used to treat a pleomorphic liposarcoma.

In some embodiments, the Hsp90 inhibitor is used to treat a liposarcoma comprising two or more of the well differentiated, dedifferentiated, myxoid, round cell, and pleomorphic subtypes.

The term “subject” as used herein, refers to an animal, typically a human (i.e., a male or female of any age group, e.g., a pediatric subject (e.g., infant, child, adolescent) or adult subject (e.g., young adult, middle-aged adult or senior adult) or other mammal, such as primates (e.g., cynomolgus monkeys, rhesus monkeys); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the subject has been the object of treatment, observation, and/or administration of the compound or drug.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, and/or turkeys.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition.

As used herein, unless otherwise specified, the terms “prevent,” “preventing” and “prevention” contemplate an action that occurs before a subject begins to suffer from the specified disease, disorder or condition, which inhibits or reduces the severity of the disease, disorder or condition.

As used herein, and unless otherwise specified, the terms “manage,” “managing” and “management” encompass preventing the recurrence of the specified disease, disorder or condition in a subject who has already suffered from the disease, disorder or condition, and/or lengthening the time that a subject who has suffered from the disease, disorder or condition remains in remission. The terms encompass modulating the threshold, development and/or duration of the disease, disorder or condition, or changing the way that a subject responds to the disease, disorder or condition.

As used herein, and unless otherwise specified, a “therapeutically effective amount” of a compound is an amount sufficient to provide a therapeutic benefit in the treatment or management of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment or management of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

In some embodiments, the Hsp90 inhibitor is a first line treatment for the liposarcoma, i.e., it is used in a subject who has not been previously administered another drug intended to treat the liposarcoma.

In other embodiments, the Hsp90 inhibitor is a second line treatment for the liposarcoma, i.e., it is used in a subject who has been previously administered another drug intended to treat the liposarcoma.

In other embodiments, the Hsp90 inhibitor is a third or fourth line treatment for the liposarcoma, i.e., it is used in a subject who has been previously administered two or three other drugs intended to treat the liposarcoma.

In some embodiments, an Hsp90 inhibitor is administered to a subject following surgical excision/removal of the liposarcoma.

In some embodiments, an Hsp90 inhibitor is administered to a subject before, during, and/or after radiation treatment of the liposarcoma.

### Hsp90 Inhibitors

Exemplary Hsp90 inhibitors include, for example, benzoquinone ansamycin Hsp90 inhibitors (e.g., as described in U.S. Patent Application Publication No. 2008/0221077), hydroquinone ansamycin Hsp90 inhibitors (e.g., as described in U.S. Pat. No. 7,282,493, U.S. Pat. No. 7,361,647, U.S. Pat. No. 7,358,370, U.S. Pat. No. 7,375,217, and U.S. Pat. No. 7,579,337), as well as non-ansamycin Hsp90 inhibitors such as SNX-2112 and analogs thereof (e.g., as described in U.S. Pat. No. 7,358,370) and NVP-AUY922 and analogs thereof.

In certain embodiments, the Hsp90 inhibitor is a hydroquinone ansamycin Hsp90 inhibitor as described in U.S. Pat. No. 7,282,493, U.S. Pat. No. 7,361,647, U.S. Pat. No. 7,358,370, U.S. Pat. No. 7,375,217, and U.S. Pat. No. 7,579,337, the disclosure of each of which is incorporated by reference in their entireties herein.

In some embodiments, the Hsp90 inhibitor is a compound of formula I:

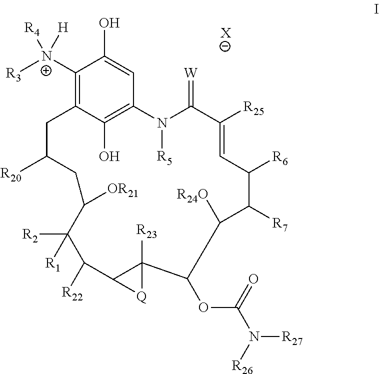

or the free base thereof,

wherein independently for each occurrence:

W is oxygen or sulfur;

Q is oxygen, NR, NC(═O)R or a bond;

X⁻ is a conjugate base of a pharmaceutically acceptable acid;

R for each occurrence is independently selected from the group consisting of hydrogen, alkyl, carbocycyl, heterocycloalkyl, aralkyl, heteroaryl, and heteroaralkyl;

R₁ is alkoxyl, —OH, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;

R₃ and R₄ are each independently selected from the group consisting of hydrogen, alkyl, alkenyl, alkynyl, aryl, carbocycyl, heterocycloalkyl, aralkyl, heteroaryl, heteroaralkyl, and —[(C(R)₂)ₚ]—R₁₆; or R₃ taken together with R₄ represent a 4-8 membered optionally substituted heterocyclic ring;

R₅ is selected from the group consisting of H, alkyl, aralkyl, and a group having the formula Ia:

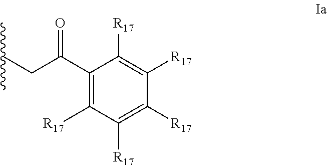

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocycloalkyl;

R₆ and R₇ are both hydrogen; or R₆ and R₇ taken together form a bond;

R₈ is hydrogen, alkyl, alkenyl, alkynyl, aryl, carbocycyl, heterocycloalkyl, aralkyl, heteroaryl, heteroaralkyl, or —[(CR₂)ₚ]—R₁₆;

R₉ is alkyl, alkenyl, alkynyl, aryl, carbocycyl, heterocycloalkyl, aralkyl, heteroaryl, heteroaralkyl, or —[(CR₂)ₚ]—R₁₆;

R₁₀ and R₁₁ are each independently selected from the group consisting of hydrogen, alkyl, alkenyl, alkynyl, aryl, carbocycyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, carbocycyl, heterocycloalkyl, aralkyl, heteroaryl, heteroaralkyl, or —[(C(R)₂)ₚ]—R₁₆;

R₁₃ and R₁₄ are each independently selected from the group consisting of hydrogen, alkyl, alkenyl, alkynyl, aryl, carbocycyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, carbocycyl, heterocycloalkyl, aralkyl, heteroaryl, and heteroaralkyl;

R₁₉ for each occurrence is independently selected from the group consisting of hydrogen, alkyl, aryl, carbocycyl, heterocycloalkyl, aralkyl, heteroaryl, and heteroaralkyl; or R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₁₈, or —CH(OR₁₈)₂;

R₂₆ and R₂₇ for each occurrence are independently selected from the group consisting of hydrogen, alkyl, aryl, carbocycyl, heterocycloalkyl, aralkyl, heteroaryl, and heteroaralkyl;

provided that when R₁ is hydroxyl, R₂ is hydrogen, R₆ and R₇ taken together form a double bond, R₂₀ is methyl, R₂₁ is methyl, R₂₂ is methyl, R₂₃ is methyl, R₂₄ is methyl, R₂₅ is methyl, R₂₆ is hydrogen, R₂₇ is hydrogen, Q is a bond, and W is oxygen; R₃ and R₄ are not both hydrogen nor when taken together represent an unsubstituted azetidine; and

the absolute stereochemistry at a stereogenic center of formula 1 may be R or S or a mixture thereof and the stereochemistry of a double bond may be E or Z or a mixture thereof.

In another embodiment, the Hsp90 inhibitor is a compound of formula II:

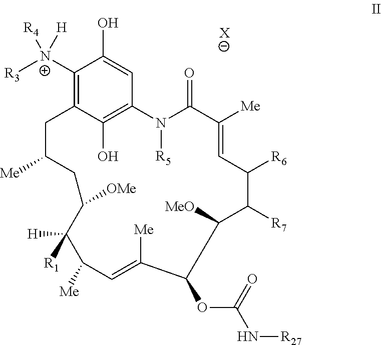

or the free base thereof, wherein independently for each occurrence:

X⁻ is selected from the group consisting of chloride, bromide, iodide, H₂PO₄⁻, HSO₄⁻, methylsulfonate, benzenesulfonate, p-toluenesulfonate, trifluoromethylsulfonate, 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate.

R₁ is hydroxyl or —OC(═O)R₈;

R₃ and R₄ are hydrogen, alkyl, alkenyl, alkynyl, carbocycyl, aralkyl, heteroaralkyl, or —[(CR₂)ₚ]—R₁₆; or R₃ taken together with R₄ represent a 4-8 membered optionally substituted heterocyclic ring;

R₅ is hydrogen or has a formula IIa:

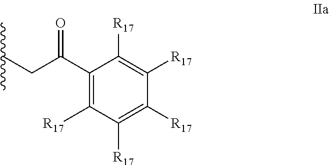

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocycloalkyl;

R₆ and R₇ are both hydrogen; or R₆ and R₇ taken together form a bond;

R₈ is hydrogen, alkyl, alkenyl, alkynyl, aryl, carbocycyl, heterocycloalkyl, aralkyl, heteroaryl, heteroaralkyl, or —[(C(R)₂)ₚ]—R₁₆;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, carbocycyl, heterocycloalkyl, aralkyl, heteroaryl, and heteroaralkyl;

R₁₉ for each occurrence is independently selected from the group consisting of hydrogen, alkyl, aryl, carbocycyl, heterocycloalkyl, aralkyl, heteroaryl, and heteroaralkyl; or R₁₈ taken together with R₁₉ represent a 4-8 membered optionally substituted ring;

R₂₇ is hydrogen, alkyl, aryl, carbocycyl, heterocycloalkyl, aralkyl, heteroaryl, or heteroaralkyl;

provided that when R₁ is hydroxyl, R₂ is hydrogen, R₆ and R₇ taken together form a double bond, R₂₇ is hydrogen; R₃ and R₄ are not both hydrogen nor when taken together represent an unsubstituted azetidine; and

the stereochemistry of a double bond may be E or Z or a mixture thereof.

In another embodiment, the Hsp90 inhibitor is a compound of formula III:

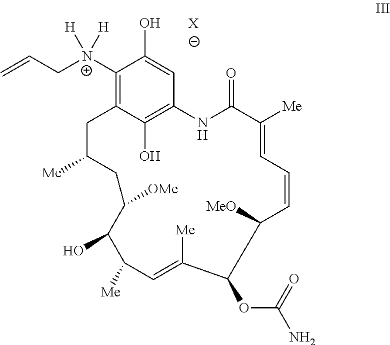

wherein X⁻ is a conjugate base of a pharmaceutically acceptable acid.

In certain embodiments, the pharmaceutically acceptable acid has a pKa between about −10 and about 3 in water. In other embodiments, the pharmaceutically acceptable acid has a pKa between about −10 and about 1 in water. In still other embodiments, the pharmaceutically acceptable acid has a pKa between about −10 and about −3 in water.

In some embodiments, X⁻ is selected from the group consisting of chloride, bromide, iodide, H₂PO₄⁻, HSO₄⁻, methylsulfonate, benzenesulfonate, p-toluenesulfonate, trifluoromethylsulfonate, 10-camphorsulfonate, naphthalene-1-sulfonic acid-5-sulfonate, ethan-1-sulfonic acid-2-sulfonate, cyclamate (i.e., N-cyclohexylsulfamate), thiocyanate, naphthalene-2-sulfonate, and oxalate.

In some embodiments, X⁻ is chloride, i.e., the Hsp90 inhibitor has the structure of formula 2:

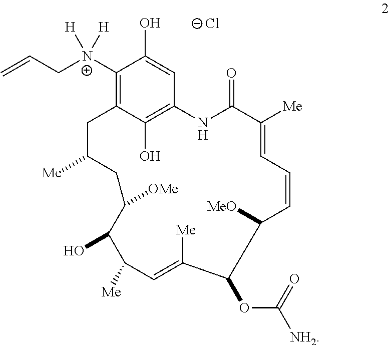

In addition, the compound of formula III (e.g., the compound of formula 2) can be co-crystallized with another acid or salt, such as an amino acid (e.g., glycine) to form a co-salt. In general, in these embodiments, the ratio of amino acid to compound can vary, but is preferably from 2:1 to 1:2.

In another embodiment, the Hsp90 inhibitor is a compound of formula IV:

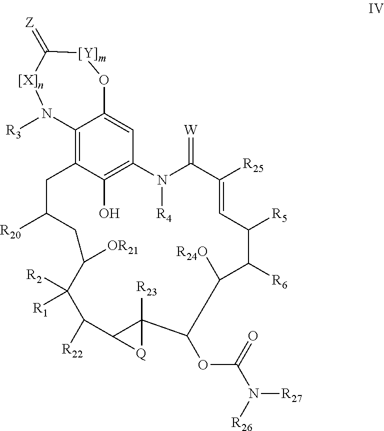

or a pharmaceutically acceptable salt thereof, wherein independently for each occurrence,

W is oxygen or sulfur;

Z is oxygen or sulfur;

Q is oxygen, NR, N(acyl) or a bond;

n is equal to 0, 1, or 2;

m is equal to 0, 1, or 2;

X and Y are independently C(R₃₀)₂; wherein R₃₀ for each occurrence is independently selected from the group consisting of hydrogen, alkyl, carbocycyl, heterocycloalkyl, aralkyl, heteroaryl, and heteroaralkyl; or —[(C(R)₂)ₚ]—R₁₆;

R for each occurrence is independently selected from the group consisting of hydrogen, alkyl, carbocycyl, heterocycloalkyl, aralkyl, heteroaryl, and heteroaralkyl;

R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;

R₃ are each independently selected from the group consisting of hydrogen, alkyl, alkenyl, alkynyl, aryl, carbocycyl, heterocycloalkyl, aralkyl, heteroaryl, heteroaralkyl, and —[(C(R)₂)ₚ]—R₁₆;

R₄ is selected from the group consisting of H, alkyl, aralkyl, and a group having the Formula IVa:

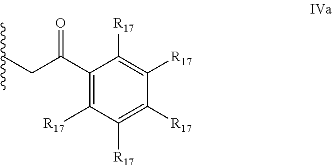

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocycloalkyl;

R₅ and R₆ are both hydrogen; or R₅ and R₆ taken together form a bond;

R₈ is hydrogen, alkyl, alkenyl, alkynyl, aryl, carbocycyl, heterocycloalkyl, aralkyl, heteroaryl, heteroaralkyl, or —[(C(R)₂)ₚ]—R₁₆;

R₉ is alkyl, alkenyl, alkynyl, aryl, carbocycyl, heterocycloalkyl, aralkyl, heteroaryl, heteroaralkyl, or —[(C(R)₂)ₚ]—R₁₆;

R₁₀ and R₁₁ are each independently selected from the group consisting of hydrogen, alkyl, alkenyl, alkynyl, aryl, carbocycyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, carbocycyl, heterocycloalkyl, aralkyl, heteroaryl, heteroaralkyl, or —[(C(R)₂)ₚ]—R₁₆;

R₁₃ and R₁₄ are each independently selected from the group consisting of hydrogen, alkyl, alkenyl, alkynyl, aryl, carbocycyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, carbocycyl, heterocycloalkyl, aralkyl, heteroaryl, and heteroaralkyl;

R₁₉ for each occurrence is independently selected from the group consisting of hydrogen, alkyl, aryl, carbocycyl, heterocycloalkyl, aralkyl, heteroaryl, and heteroaralkyl; or R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₁₈, or —CH(OR₁₈)₂;

R₂₆ and R₂₇ for each occurrence are independently selected from the group consisting of hydrogen, alkyl, aryl, carbocycyl, heterocycloalkyl, aralkyl, heteroaryl, and heteroaralkyl; and

the absolute stereochemistry at a stereogenic center of formula IV may be R or S or a mixture thereof and the stereochemistry of a double bond may be E or Z or a mixture thereof.

In another embodiment, the Hsp90 inhibitor is a compound of formula V:

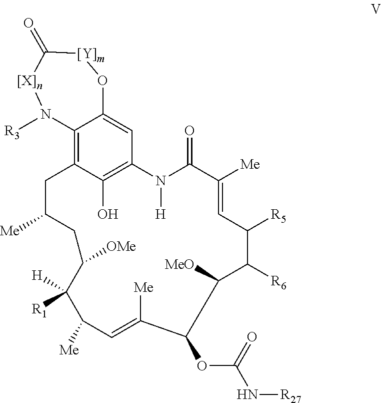

or a pharmaceutically acceptable salt thereof, wherein independently for each occurrence:

n is equal to 0, 1, or 2;

m is equal to 0, 1, or 2;

X and Y are independently C(R₃₀)₂; wherein R₃₀ for each occurrence is independently selected from the group consisting of hydrogen, alkyl, carbocycyl, heterocycloalkyl, aralkyl, heteroaryl, and heteroaralkyl; or —[(CR₂)ₚ]—R₁₆;

R₁ is hydroxyl or —OC(O)R₈;

R₃ is hydrogen, alkyl, alkenyl, alkynyl, carbocycyl, aralkyl, heteroaralkyl, or —[(CR₂)ₚ]—R₁₆;

R₅ and R₆ are both hydrogen; or R₅ and R₆ taken together form a bond;

R₈ is hydrogen, alkyl, alkenyl, alkynyl, aryl, cycloalkyl, heterocarbocycyl, aralkyl, heteroaryl, heteroaralkyl, or —[(C(R)₂)ₚ]—R₁₆;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocarbocycyl, aralkyl, heteroaryl, and heteroaralkyl;

R₁₉ for each occurrence is independently selected from the group consisting of hydrogen, alkyl, aryl, carbocycyl, heterocycloalkyl, aralkyl, heteroaryl, and heteroaralkyl; or R₁₈ taken together with R₁₉ represent a 4-8 membered optionally substituted ring;

R₂₇ is hydrogen, alkyl, aryl, carbocycyl, heterocycloalkyl, aralkyl, heteroaryl, or heteroaralkyl; and

the stereochemistry of a double bond may be E or Z or a mixture thereof.

In certain embodiments, the Hsp90 inhibitor is:

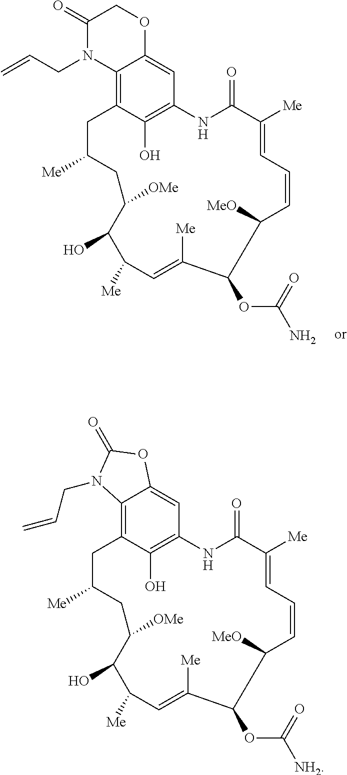

In certain embodiments, the Hsp90 inhibitor is a benzoqinone ansamycin Hsp90 inhibitor.

For example, in some embodiments, the Hsp90 inhibitor is a compound of formula VI:

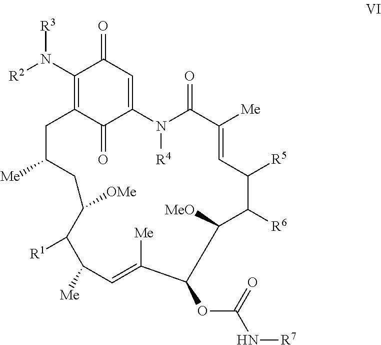

or a pharmaceutically acceptable salt thereof, wherein;

R¹ is H, —OR⁸, —SR⁸—N(R⁸)(R⁹), —N(R⁸)C(O)R⁹, —N(R⁸)C(O)OR⁹, —N(R⁸)C(O)N(R⁸)(R⁹),

### —OC(O)R⁸, —OC(O)OR⁸, —OS(O)₂R⁸, —OS(O)₂OR⁸, —OP(O)₂OR⁸, CN or ═O;

each of R² and R³ independently is H, alkyl, alkenyl, alkynyl, carbocycyl, cycloalkenyl, heterocycloalkyl, aryl, aralkyl, heteroaryl, heteroaralkyl, —C(═O)CH₃ or) —[(C(R¹⁰)₂)ₚ]—R¹¹; or R² and R³ taken together with the nitrogen to which they are bonded represent a 3-8 membered optionally substituted heterocyclic ring which contains 1-3 heteroatoms selected from O, N, S, and P;

p independently for each occurrence is 0, 1, 2, 3, 4, 5, or 6;

R⁴ is H, alkyl, akenyl, or aralkyl;

R⁵ and R⁶ are each H; or R⁵ and R⁶ taken together form a bond;

R⁷ is hydrogen alkyl, alkenyl, alkynyl, carbocycyl, cycloalkenyl, heterocycloaklyl, aryl, aralkyl, heteroaryl, heteroaralkyl, or —[(C(R¹⁰)₂)ₚ]—R¹¹;

each of R⁸ and R⁹ independently for each occurrence is H, alkyl, alkenyl, alkynyl, carbocycyl, cycloalkenyl, heterocycloaklyl, aryl, aralkyl, heteroaryl, heteroaralkyl, or —[(C(R¹⁰)₂)ₚ]—R¹¹; or R⁸ and R⁹ taken together represent a 3-8 membered optionally substituted heterocyclic ring which contains 1-3 heteroatoms selected from O, N, S, and P;

R¹⁰ for each occurrence independently is H, alkyl, alkenyl, alkynyl, carbocycyl, cycloalkenyl, heterocycloaklyl, aryl, aralkyl, heteroaryl, or heteroaralkyl; and

R¹¹ for each occurrence independently is H, carbocycyl, aryl, heteroaryl, heterocycloalkyl,

—OR⁸, —SR⁸, —N(R⁸)(R⁹), —N(R⁸)C(O)R⁹, —N(R⁸)C(O)OR⁹, —N(R⁸)C(O)N(R⁸)(R⁹), —OC(O)R⁸, —OC(O)OR⁸, —OS(O)₂R⁸, —OS(O)₂OR⁸, —OP(O)₂OR⁸, —C(O)R⁸, —C(O)₂R⁸, —C(O)N(R⁸)(R⁹), halide, or CN.

Examples of benzoquinone ansamycin Hsp90 inhibitors encompassed by formula VI include, but are not limited to:

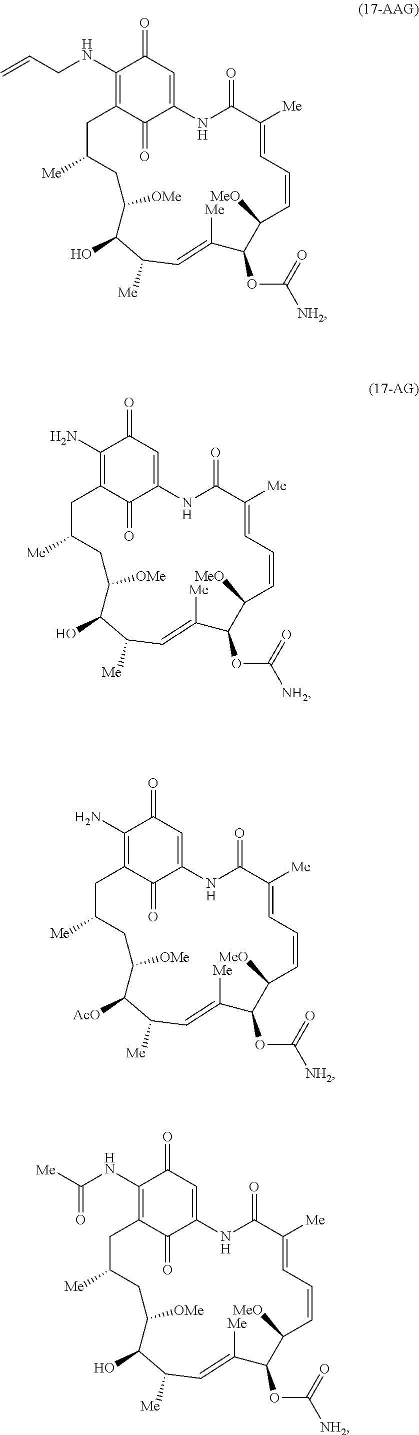

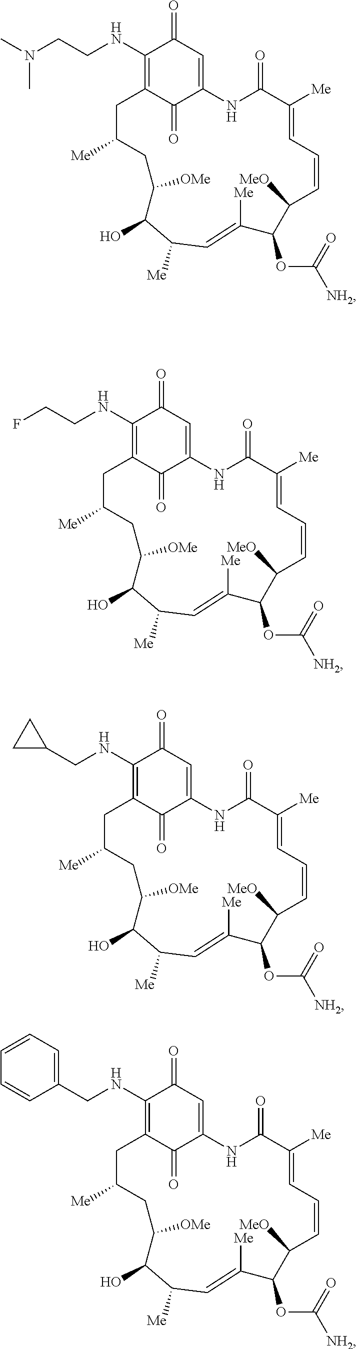

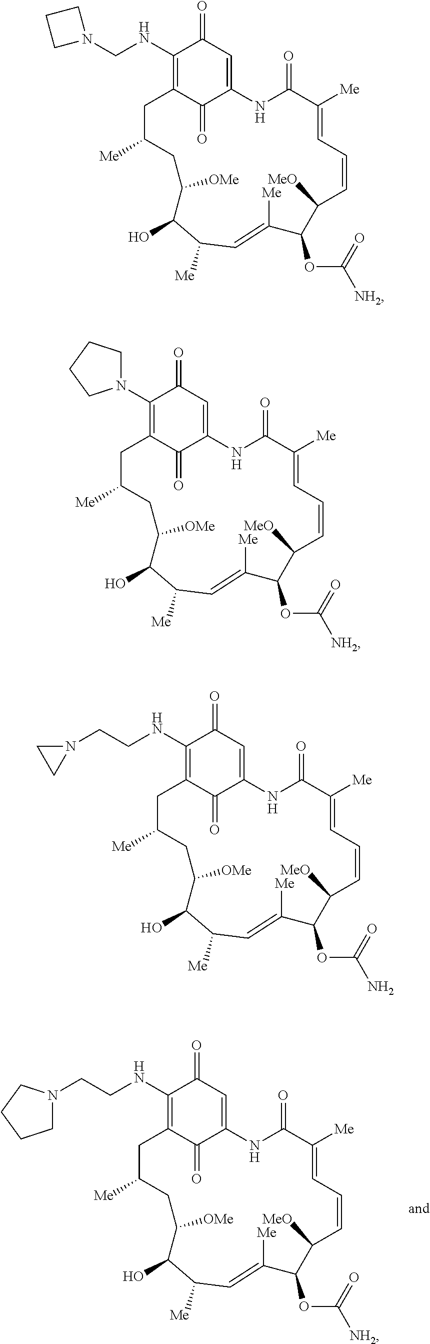

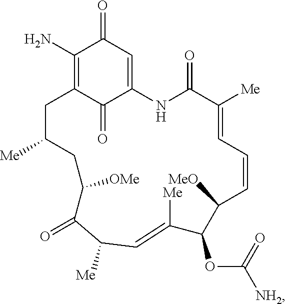

and pharmaceutically acceptable salts thereof.

In some embodiments, the Hsp90 inhibitor is 17-AG.

Exemplary novel formulations of substantially amorphous 17-AG and other substantially amorphous benzoquinone ansamycins are described in U.S. Patent Application Publication No. 2008/0255080, the disclosure of which is incorporated by reference in its entirety herein.

In certain embodiments, the Hsp90 inhibitor is substantially amorphous 17-AG.

In certain embodiments, the substantially amorphous 17-AG is provided in a solid molecular dispersion. In certain embodiments, the substantially amorphous 17-AG is provided in a solid molecular dispersion which further comprises a crystallization inhibitor.

In certain embodiments, the substantially amorphous 17-AG is administered in a pharmaceutical composition. In certain embodiments, the pharmaceutical composition further comprises a crystallization inhibitor. In certain embodiments, the pharmaceutical composition may be in the form of a paste, solution, slurry, ointment, suspension, emulsion or solid dispersion.

The term “molecular dispersion” as used herein refers to a type of dispersion (e.g., solid or liquid dispersion) wherein one component is dispersed throughout another component such that the system is chemically and physically uniform and homogeneous throughout. These systems are determined to be substantially amorphous as evidenced by thermal analysis (e.g., differential scanning calorimetry), diffractive (e.g., X-ray diffraction), or imaging (e.g., polarized light microscopy) techniques.

The term “substantially amorphous” as used herein means that the majority of the compound present in a composition is present in amorphous form and that the composition has less than about 20% crystalline compound, less than about 15% crystalline compound, less than about 10% crystalline compound, less than about 5% crystalline compound, less than about 3% crystalline compound, or less than about 1% crystalline compound, less than about 0.1% crystalline compound, or less than about 0.01% crystalline compound. In some embodiments of the present invention, the compound present in a composition contains no detectable crystalline material.

The term “crystallization inhibitor” means a pharmaceutically acceptable excipient which substantially inhibits the conversion of a compound from the amorphous form to one or more crystalline forms in the solid state or in solution. A crystallization inhibitor may also substantially inhibit crystal growth in the gastrointestinal tract for long enough (e.g., about 1 to 6 hours) to allow for enhanced absorption of at least 50%, over conventional delivery, of the compound into the bloodstream.

Exemplary crystallization inhibitors include, but are not limited to, polyvinylpyrrolidone (PVP) (including homo- and copolymers of polyvinylpyrrolidone and homopolymers or copolymers of N-vinylpyrrolidone); crospovidone; gums; cellulose derivatives (including hydroxypropyl methylcellulose (HPMC), hydroxypropyl methylcellulose phthalate, hydroxypropyl cellulose, ethyl cellulose, hydroxyethylcellulose, sodium carboxymethyl cellulose, calcium carboxymethyl cellulose, sodium carboxymethyl cellulose, and others); dextran; acacia; homo- and copolymers of vinyllactam, and mixtures thereof; cyclodextrins; gelatins; hypromellose phthalate; sugars; polyhydric alcohols; polyethylene glycol (PEG); polyethylene oxides; polyoxyethylene derivatives; polyvinyl alcohol; propylene glycol derivatives and the like, SLS, Tween, Eudragit; and combinations thereof. The crystallization inhibitor may be water soluble or water insoluble.

HPMC polymers vary in the chain length of their cellulosic backbone and consequently in their viscosity as measured for example at a 2% (w/w) in water. In certain embodiments, the HPMC polymer has a viscosity in water (at a concentration of 2% (w/w)), of about 100 to about 100,000 cP, about 1000 to about 15,000 cP, for example about 4000 cP. In certain embodiments, the molecular weight of the HPMC polymer has greater than about 10,000, but not greater than about 1,500,000, not greater than about 1,000,000, not greater than about 500,000, or not greater than about 150,000.

HPMC polymers also vary in the relative degree of substitution of available hydroxyl groups on the cellulosic backbone by methoxy and hydroxypropoxy groups. With increasing hydroxypropoxy substitution, the resulting HPMC polymer becomes more hydrophilic in nature. In certain embodiments, the HPMC polymer has about 15% to about 35%, about 19% to about 32%, or about 22% to about 30%, methoxy substitution, and having about 3% to about 15%, about 4% to about 12%, or about 7% to about 12%, hydroxypropoxy substitution.

Exemplary HPMC polymers include, but are not limited to, hydroxypropylmethylcellulose (HPMC), hydroxypropylmethylcellulose acetate phthalate (HPMC-AP), hydroxypropylmethylcellulose acetate succinate (HPMC-AS), hydroxypropylmethylcellulose acetate trimellitate (HPMC-AT) and hydroxypropylmethylcellulose phthalate (HPMC-P).

Grades of hydroxypropylmethylcellulose (HPMC) include, but are not limited to, 3FG, 4FG, 5FG, 6FG, 15FG, 50FG and K100M. Grades of hydroxypropylmethylcellulose acetate succinate (HPMC-AS) include HPMC-AS-LF, HPMC-AS-MF, HPMC-AS-HF, HPMC-AS-LG, HPMC-AS-MG and HPMC-AS-HG. Grades of hydroxypropyl-methylcellulose phthalate (HPMC-P) include 50, 55, 55S.

In certain embodiments, the HPMC polymer is an HPMC-AS polymer. In certain embodiments, the HPMC polymer is an HPMC-AS-LG polymer. In certain embodiments, the HPMC polymer is an HPMC-AS-MG polymer. In certain embodiments, the HPMC polymer is an HPMC-AS-HG polymer. In certain embodiments, the HPMC polymer is an HPMC-P polymer.

Other exemplary HPMC polymers are available under the brand names Methocel™ of Dow Chemical Co. and Metolose™ of Shin-Etsu Chemical Co. Examples of suitable HPMC polymers having medium viscosity include Methocel™ E4M, and Methocel™ K4M, both of which have a viscosity of about 4000 cP at 2% (w/w) water. Examples of HPMC polymers having higher viscosity include Methocel™ E10M, Methocel™ K15M, and Methocel™ K100M, which have viscosities of about 10,000 cP, 15,000 cP, and 100,000 cP respectively viscosities at 2% (w/w) in water.

In certain embodiments, a crystallization inhibitor employed by the present invention is a PVP polymer.

In certain embodiments, PVP polymers employed in the present invention have a molecular weight of about 2,500 to about 3,000,000 Daltons, about 8,000 to about 1,000,000 Daltons, about 10,000 to about 400,000 Daltons, about 10,000 to about 300,000 Daltons, about 10,000 to about 200,000 Daltons, about 10,000 to about 100,000 Daltons, about 10,000 to about 80,000 Daltons, about 10,000 to about 70,000 Daltons, about 10,000 to about 60,000 Daltons, about 10,000 to about 50,000 Daltons, or about 20,000 to about 50,000 Daltons.

In certain embodiments, PVP polymers employed in the present invention have a dynamic viscosity, 10% in water at 20° C., of about 1.3 to about 700, about 1.5 to about 300, or about 3.5 to about 8.5 mPas.

In certain embodiments, PVP polymers employed in the present invention are selected from PVP K-30, PVP K-90 and Kollidon® PVP polymers (e.g., Kollidon®12PF, Kollidon®17PF, Kollidon®25, Kollidon®30, Kollidon®90F, and Kollidon VA 64 (copolyvidone)).

In certain embodiments, a crystallization inhibitor employed by the present invention is a PEG polymer.

In certain embodiments, PEG polymers employed in the present invention have has an average molecular about 5,000-20,000 Dalton, about 5,000-15,000 Dalton, or about 5,000-10,000 Dalton.

In certain embodiments, a crystallization inhibitor employed by the present invention is a surfactant. In certain embodiments, the crystallization inhibitor is a Tween® surfactant. Exemplary Tweens® include Tween®20, Tween®40, Tween®60, Tween®65 and Tween®80.

### Combination Therapy

As generally defined above, in certain embodiments, the Hsp90 inhibitor (e.g., a compound of formula I, II, III, IV, V, VI, or sub-genera thereof) is further used in combination with an additional anti-cancer agent.

By “in combination with,” it is not intended to imply that the Hsp90 inhibitor and additional anti-cancer agent must be administered at the same time and/or formulated for delivery together, although these methods of delivery are certainly within the scope of the invention. The Hsp90 inhibitor can be administered concurrently, prior to or subsequent to, one or more additional anti-cancer agents. In general, the Hsp90 inhibitor and the anti-cancer agent will be administered at a dose and/or on a time schedule determined for each. In will further be appreciated that the Hsp90 inhibitor and the additional anti-cancer agent utilized in this combination may be administered together in a single pharmaceutical composition or administered separately in different pharmaceutical compositions. The particular combination to employ in a regimen will take into account compatibility of the Hsp90 inhibitor with the additional anti-cancer agent and/or the desired therapeutic effect to be achieved.

In certain embodiments, the Hsp90 inhibitor and the additional anti-cancer agent are administered concurrently (i.e., administration of the two agents at the same time or day, or within the same treatment regimen) or sequentially (i.e., administration of one agent over a period of time followed by administration of the other agent for a second period of time, or within different treatment regimens).

In certain embodiments, the Hsp90 inhibitor and the additional anti-cancer agent are administered concurrently. For example, in certain embodiments, the Hsp90 inhibitor and the additional anti-cancer agent are administered at the same time. In certain embodiments, the Hsp90 inhibitor and the additional anti-cancer agent are administered on the same day. In certain embodiments, the Hsp90 inhibitor and the additional anti-cancer agent are administered within the same treatment regimen. In certain embodiments, the Hsp90 inhibitor is administered after the additional anti-cancer agent on the same day or within the same treatment regimen. In certain embodiments, the Hsp90 inhibitor is administered before the additional anti-cancer agent on the same day or within the same treatment regimen.

In certain embodiments, an Hsp90 inhibitor is concurrently administered with additional anti-cancer agent for a period of time, after which point treatment with the additional anti-cancer agent is stopped and treatment with the Hsp90 inhibitor continues.

In other embodiments, an Hsp90 inhibitor is concurrently with the additional anti-cancer agent for a period of time, after which point treatment with the Hsp90 inhibitor is stopped and treatment with the additional anti-cancer agent continues.

In certain embodiments, the Hsp90 inhibitor and the additional anti-cancer agent are administered sequentially. For example, in certain embodiments, the Hsp90 inhibitor is administered after the treatment regimen of the additional anti-cancer agent has ceased. In certain embodiments, the additional anti-cancer agent is administered after the treatment regimen of the Hsp90 inhibitor has ceased.

Exemplary anti-cancer agents, include, but are not limited to, radiation therapy, interferon (e.g., interferon α, interferon γ), antibodies (e.g. HERCEPTIN (trastuzumab), T-DM1, AVASTIN (bevacizumab), ERBITUX (cetuximab), VECTIBIX (panitumumab), RITUXAN (rituximab) BEXXAR (tositumomab)), anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel, albumin-bound paclitaxel (ABRAXANE), nab-paclitaxel, docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase Inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lap atinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK®), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

In certain embodiments the anti-cancer agent is selected from radiation therapy, cyclophosphamide, ifosfamide, dacarbazine, doxorubicin, gemcitabine, docetaxel, paclitaxel, irinotecan, carboplatin and trabectedin.

### Pharmaceutical Compositions and Administration

Also provided herein for use in the present invention are pharmaceutical compositions comprising an Hsp90 inhibitor (e.g., a compound of formula I, II, III, IV, V or VI, or subgenera thereof) and a pharmaceutically acceptable excipient.

Pharmaceutically acceptable excipients include any and all solvents, diluents, or other liquid vehicle, surface active agents, isotonic agents, thickening or emulsifying agents, sugars, polymers, surfactants, antioxidants, solubilizing or suspending agents, metal chelators, preservatives, dilutents, granulating and/or dispersing agents, binding agents, and/or lubricating agents, or combinations thereof, as suited to the particular dosage form desired and according to the judgment of the formulator. Remington's Pharmaceutical Sciences, Sixteenth Edition, E. W. Martin (Mack Publishing Co., Easton, Pa., 1980) discloses various carriers used in preparing pharmaceutically acceptable pharmaceutical compositions and known techniques for the preparation thereof. Except insofar as any conventional carrier medium is incompatible with the inventive compositions, such as by producing any undesirable biological effect or otherwise interacting in a deleterious manner with any component of the composition, its use is contemplated to be within the scope of this invention.

In certain embodiments, at least one excipient provided in the pharmaceutical composition is a sugar. The term “sugar” as used herein refers to a natural or an unnatural monosaccharide, disaccharide, oligosaccharide, or polysaccharide, comprising one or more triose, tetrose, pentose, hexose, heptose, octose, or nonose saccharides. Sugars may include substances derived from saccharides by reduction of the carbonyl group (alditols), by oxidation of one or more terminal groups to carboxylic acids (aldonic acids), or by replacement of one or more hydroxyl group(s) by a hydrogen (deoxy sugars), an amino group (amino sugars), a thiol group (thio sugars), an acylamino group, a sulfate group, a phosphate group, or similar heteroatomic group; or any combination of the foregoing modifications. The term sugar also includes derivatives of these compounds (i.e., sugars that have been chemically modified by acylation, alkylation, and formation of glycosidic bonds by reaction of sugar alcohols with aldehydes or ketones, etc.). Sugars may be present in cyclic form (i.e., oxiroses, oxetosesm furanoses, pyranoses, septanoses, octanoses, etc.) as hemiacetals, hemiketals, or lactones, or in acyclic form. The saccharides may be ketoses, aldoses, polyols and/or a mixture of ketoses, aldoses and polyols.

Exemplary sugars include, but are not limited to, glycerol, polyvinylalcohol, propylene glycol, sorbitol, ribose, arabinose, xylose, lyxose, allose, altrose, mannose, mannitol, gulose, dextrose, idose, galactose, talose, glucose, fructose, dextrates, lactose, sucrose, starches (i.e., amylase and amylopectin), sodium starch glycolate, cellulose and cellulose derivatives (i.e., methylcellulose, hydroxypropyl cellulose, hydroxyethyl cellulose, hydroxyethylmethyl cellulose, carboxymethyl cellulose, cellulose acetate, cellulose acetate phthalate, croscarmellose, hypomellose, and hydroxypropyl methyl cellulose), carrageenan, cyclodextrins (e.g., hydroxypropyl-gamma-CD), dextrin, polydextrose, and trehalose. In certain embodiments, the sugar is selected from anhydrous lactose, lactose monohydrate, trehalose and hydroxypropyl-gamma-CD.

In certain embodiments, at least one excipient provided in the pharmaceutical composition is a polymer. Exemplary polymers include, but are not limited to, polyvinyl alcohol (PVA), getaLin, polyvinyl pyrolidone (PVP), albumin, polyethyleneimine (PEI), acacia gum, cellulose derivatives, calcium polypectate, maleic anhydride derivatives, polyacrylic and methacrylic acid, phospholipids, glycols (such as propylene glycol or polyethylene glycol), polyglycolide and lactide derivatives, polyethylene-polyoxypropylene-block polymers, starch, waxes, oils, alginates and alginic acid, calcium caseinate, carrageenan, pectins, polyhexametaphosphate, polyvinyl acetate, polyvinyl alcohol, and the like; mixtures thereof and the like. In certain embodiments, the polymer is polyvinyl alcohol (PVA).

In certain embodiments, at least one excipient provided in the pharmaceutical composition is a surfactant. Exemplary surfactants include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the surfactant is a Tween surfactant (e.g., Tween 60, Tween 80, etc.).

In certain embodiments, at least one excipient provided in the pharmaceutical composition is an antioxidant. Exemplary antioxidants include, but are not limited to, alpha tocopherol, ascorbic acid, acorbyl palmitate, butylated hydroxyanisole, butylated hydroxytoluene, monothioglycerol, lecithin, potassium metabisulfite, propionic acid, propyl gallate, sodium ascorbate, sodium bisulfite, sodium metabisulfite, sodium sulfite, thioglycerol, cysteine hydrochloride, thioglycerol, sodium mercaptoacetate, sodium formaldehyde sulfoxylate (SFS), lecithin and organic phosphites (e.g., dimethyl phosphite, diethyl phosphite, dibutyl phosphite, triethyl phosphite, tris(2-chloroethyl)phosphite, tris (2-4-t-butyl-phenyl)-phosphite, etc.). In some embodiments, the antioxidant is ascorbate and/or a salt or hydrate thereof. In certain embodiments, the antioxidant is L-ascorbic acid.

In certain embodiments, at least one excipient provided in the pharmaceutical composition is a solubilizing or suspending agent. Exemplary solubilizing or suspending agents include, but are not limited to, water, organic solvents and oils, or mixtures thereof.

Exemplary organic solvents include, but are not limited to, ethanol, propanol, butanol, chloroform, dichloromethane, ethyl acetate, diethyl ether, hexames, acetone, benzene, toluene, and xylenes.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In certain embodiments, at least one excipient provided in the pharmaceutical composition is a metal chelator. Exemplary metal chelators include, but are not limited to, ethylenediaminetetraacetic acid (EDTA), citric acid monohydrate, disodium edetate, dipotassium edetate, edetic acid, fumaric acid, malic acid, phosphoric acid, sodium edetate, tartaric acid, trisodium edentate, DTPA (diethylene-triamine-penta-acetic acid) and its salt, EGTA (ethylene glycol tetraacetic acid), NTA (nitriloacetic acid), sorbitol, tartaric acid, N-hydroxy iminodiacetate, hydroxyethyl-ethylene diamine-tetraacetic acid, 1- and 3-propanediamine tetra acetic acid, 1- and 3-diamino-2-hydroxy propane tetra-acetic acid, sodium gluconate, hydroxy ethane diphosphonic acid, phosphoric acid, and salts and hydrates thereof. In some embodiments, the metal chelator is EDTA or a salt and/or hydrate thereof. In certain embodiments, the metal chelator is ethylenediamine tetraacetic acid disodium calcium salt dihydrate.

In certain embodiments, at least one excipient provided in the pharmaceutical composition is a preservative.

Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include, but are not limited to, ethanol, polyethylene glycol, phenol, phenolic compounds, bisphenol, chlorobutanol, hydroxybenzoate, and phenylethyl alcohol.

Exemplary acidic preservatives include, but are not limited to, vitamin A, vitamin C, vitamin E, beta-carotene, citric acid, acetic acid, dehydroacetic acid, ascorbic acid, sorbic acid, and phytic acid.

Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

In certain embodiments, at least one excipient provided in the pharmaceutical composition is a diluent. Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

In certain embodiments, at least one excipient provided in the pharmaceutical composition is a granulating and/or dispersing agent. Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

In certain embodiments, at least one excipient provided in the pharmaceutical composition is a binding agent. Exemplary binding agents include, but are not limited to, starch (e.g. cornstarch and starch paste); gelatin; sugars (e.g. sucrose, glucose, dextrose, dextrin, molasses, lactose, lactitol, mannitol, etc.); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

In certain embodiments, at least one excipient provided in the pharmaceutical composition is a buffering agent. The buffering agent should buffer the pharmaceutical composition between a pH of about 1 to about 5, or between a pH of about 1.8 to about 3.5, or between a pH of about 3 to about 3.3. Exemplary buffering agents include, but are not limited to, citrate buffer solutions, acetate buffer solutions, phosphate buffer solutions, ammonium chloride, calcium carbonate, calcium chloride, calcium citrate, citric acid monohydrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof. In certain embodiments, the buffering agent is citric acid monohydrate.

In certain embodiments, at least one excipient provided in the pharmaceutical composition is a lubricating agent. Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

In some embodiments, pharmaceutical composition comprises an antioxidant and a metal chelator. In other embodiments, pharmaceutical composition comprises an antioxidant and a buffering agent. In other embodiments, pharmaceutical composition comprises an antioxidant, a buffering agent and a metal chelator.

In certain embodiments, pharmaceutical composition comprises a metal chelator. In some embodiments, pharmaceutical composition comprises a metal chelator and an antioxidant. In other embodiments, the pharmaceutical composition comprises a metal chelator and a buffering agent. In some embodiments, the pharmaceutical composition comprises a metal chelator, an antioxidant and a buffering agent.

In certain embodiments, pharmaceutical composition comprises a buffering agent and an antioxidant. In other embodiments, the pharmaceutical composition comprises a buffering agent and a metal chelator. In some embodiments, pharmaceutical composition comprises a buffering agent, an antioxidant and a metal chelator.

In some embodiments, the antioxidant is ascorbate or a salt and/or hydrate thereof, the metal chelator is ethylenediamine tetraacetic acid (EDTA) or a salt and/or hydrate thereof, and the buffering agent is citrate or a salt and/or hydrate thereof. In certain embodiments, the antioxidant is L-ascorbic acid, the metal chelator is ethylenediamine tetraacetic acid disodium calcium dihydrate, and the buffering agent is citric acid monohydrate.

In certain embodiments, the molar ratio of EDTA (e.g., EDTA disodium calcium) to compound of formula I (e.g., the compound of formula 2) is in the range from about 0.001 to about 0.1, or from about 0.01 to about 0.05. In certain embodiments, the molar ratio of ascorbic acid (e.g., L-ascorbic acid) to compound of formula I (e.g., the compound of formula 2) is in the range from about 0.001 to about 1, or from 0.01 to about 1. In certain embodiments, the molar ratio of citrate (e.g., citric acid monohydrate) to compound of formula I (e.g., the compound of formula 2) is in the range of about 0.05 to about 2, or from 0.2 to about 1.

In some embodiments, the molar ratio of EDTA (e.g., EDTA disodium calcium) to compound of formula I (e.g., the compound of formula 2) is in the range from about 0.001 to about 0.1, and the molar ratio of ascorbic acid (e.g., L-ascorbic acid) to compound of formula I is in the range from about 0.001 to about 1. In certain embodiments, the molar ratio of EDTA (e.g., EDTA disodium calcium) to said compound of formula I (e.g., the compound of formula 2) is in the range from about 0.01 to about 0.05, and the molar ratio of ascorbic acid (e.g., L-ascorbic acid) to compound of formula I (e.g., the compound of formula 2) is in the range from about 0.01 to about 1.

In some embodiments, the molar ratio of EDTA (e.g., EDTA disodium calcium) to compound of formula I (e.g., the compound of formula 2) is in the range from about 0.001 to about 0.1, the molar ratio of ascorbic acid (e.g., L-ascorbic acid) to compound of formula I (e.g., the compound of formula 2) is in the range from about 0.001 to about 1, and the molar ratio of citrate (e.g., citric acid monohydrate) to compound of formula I (e.g., the compound of formula 2) is in the range of about 0.05 to about 2. In certain embodiments, the molar ratio of EDTA (e.g., EDTA disodium calcium) to compound of formula I (e.g., the compound of formula 2) is in the range from about 0.01 to about 0.05, the molar ratio of ascorbic acid (e.g., L-ascorbic acid) to compound of formula I (e.g., the compound of formula 2) is in the range from about 0.01 to about 1, and the molar ratio of citrate (e.g., citric acid monohydrate) to compound of formula I e.g., the compound of formula 2) is in the range of about 0.2 to about 1.

In some embodiments, the compound of formula I (e.g., the compound of formula 2) is present at a concentration of about 0.00016 M to about 0.160 M. In certain embodiments, the compound of formula I (e.g., the compound of formula 2) is present at a concentration of about 0.00032 M to about 0.080 M.

An example of a suitable aqueous formulation of a compound of formula I (e.g., the compound of formula 2) is an aqueous buffer containing citric acid (from about 5 mM to about 250 mM, preferably from about 25 mM to about 150 mM), ascorbic acid (from about 0.1 mM to about 250 mM, preferably from about 0.1 mM to about 50 mM), and calcium-disodium ethylenediamine tetraacetic acid (from about 0.2 mM to about 20 mM, or from about 1 mM to about 3 mM) with the pH being adjusted to about 3.1 with sodium hydroxide. The components of the formulation act as buffering agent, anti-oxidant and metal chelator, respectively.

Examples of suitable pharmaceutical compositions comprising compounds of formula I (e.g., the compound of formula 2) are disclosed in U.S. Pat. Nos. 7,282,493, 7,361,647, and 7,375,217 and U.S. Patent Application Publication No. 2008/0255080, the disclosures of which are incorporated by reference in their entireties herein.

In some embodiments, the one or more pharmaceutically acceptable excipients added to the pharmaceutical composition are at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

The pharmaceutical compositions described herein may be prepared by any method known or hereafter developed in the art of pharmacology. In general, such preparatory methods include the step of bringing the inventive composition into association with one or more excipients as described above and herein, and then, if necessary and/or desirable, shaping and/or packaging the product into a desired single- or multi-dose unit.

A pharmaceutical composition of the present invention may be prepared, packaged, and/or sold in bulk, as a single unit dose, and/or as a plurality of single unit doses. As used herein, a “unit dose” is discrete amount of the pharmaceutical composition comprising a predetermined amount of the inventive composition.

The relative amounts of the active ingredient(s) and excipients in the pharmaceutical composition will vary, depending upon the identity, size, and/or condition of the subject treated and further depending upon the route by which the pharmaceutical composition is to be administered.

The mode of administration of the active ingredients and/or pharmaceutical composition includes, but is not limited to, oral administration, parenteral administration, intravenous administration, sublingual administration, ocular administration, transdermal administration, intradermal administration, rectal administration, vaginal administration, topical administration, intramuscular administration, intraarterial administration, intrathecal administration, subcutaneous administration, pulmonary administration or intranasal administration. In certain embodiments, the mode of administration is intravenous administration. In other embodiments, the mode of administration is oral administration.

Liquid dosage forms for oral and parenteral administration include, but are not limited to, pharmaceutically acceptable emulsions, microemulsions, solutions, suspensions, syrups and elixirs. In addition to the inventive composition, the liquid dosage form may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the inventive compositions are mixed with solubilizing agents such as Cremophor, alcohols, oils, modified oils, glycols, polysorbates, cyclodextrins, polymers, and combinations thereof.

Injectable pharmaceutical compositions, for example, sterile injectable aqueous or oleaginous suspensions may be prepared according to the known art using suitable dispersing or wetting agents and suspending agents. The sterile injectable pharmaceutical composition may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable pharmaceutical compositions can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Pharmaceutical compositions for rectal or vaginal administration are typically suppositories which can be prepared by mixing the inventive compositions with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the inventive composition is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid dosage forms of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the inventive composition only, or preferentially, in a certain part of the intestinal tract, optionally, in a delayed manner. Examples of embedding inventive compositions which can be used include polymeric substances and waxes. Solid dosage pharmaceutical compositions of a similar type may be employed as fillers in soft and hard-filled gelatin capsules using such excipients as lactose or milk sugar as well as high molecular weight polyethylene glycols and the like.

Pharmaceutical compositions according to the invention can be provided in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the inventive composition may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Pharmaceutical compositions for topical and/or transdermal administration of an inventive composition includes ointments, pastes, creams, lotions, gels, powders, solutions, sprays, inhalants and/or patches. Generally, the inventive composition is admixed under sterile conditions with a pharmaceutically acceptable carrier and/or any needed preservatives and/or buffers as may be required. The present invention also contemplates the use of transdermal patches, which often have the added advantage of providing controlled delivery of an active ingredient to the body. Such dosage forms may be prepared, for example, by dissolving and/or dispensing the inventive composition in the proper medium. Alternatively or additionally, the rate may be controlled by either providing a rate controlling membrane and/or by dispersing the inventive composition in a polymer matrix and/or gel.

Suitable devices for use in delivering intradermal pharmaceutical compositions include short needle devices such as those described in U.S. Pat. Nos. 4,886,499; 5,190,521; 5,328,483; 5,527,288; 4,270,537; 5,015,235; 5,141,496; and 5,417,662. Intradermal pharmaceutical compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Pharmaceutical compositions suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable pharmaceutical compositions may, for example, comprise from about 1% to about 10% (w/w) of the inventive composition. Pharmaceutical compositions for topical administration may further comprise one or more of the additional ingredients described above and herein.

A pharmaceutical composition may be prepared, packaged, and/or sold for pulmonary administration via the buccal cavity. Such a pharmaceutical composition may comprise dry particles which comprise the inventive composition and which have a diameter in the range from about 0.5 to about 7 nanometers or from about 1 to about 6 nanometers. Such pharmaceutical compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the inventive composition. The propellant may further comprise additional ingredients such as a liquid non-ionic and/or solid anionic surfactant and/or a solid diluent (which may have a particle size of the same order as particles comprising the active ingredient).

Pharmaceutical compositions for pulmonary administration may provide the inventive composition in the form of droplets of a solution and/or suspension. Such pharmaceutical compositions may be prepared, packaged, and/or sold as aqueous and/or dilute alcoholic solutions and/or suspensions, and may conveniently be administered using any nebulization and/or atomization device. Such pharmaceutical compositions may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

The pharmaceutical compositions described herein as being useful for pulmonary administration are also useful for intranasal administration. Another pharmaceutical composition suitable for intranasal administration is a coarse powder comprising inventive composition and having an average particle from about 0.2 to 500 micrometers. Such a pharmaceutical composition is administered in the manner in which snuff is taken, i.e. by rapid inhalation through the nasal passage from a container of the powder held close to the nares. Pharmaceutical compositions suitable for nasal administration may, for example, comprise from about as little as 0.1% (w/w) and as much as 100% (w/w) of the inventive composition, and may comprise one or more of the additional ingredients as described above and herein.

Actual dosage levels of the active ingredients in the pharmaceutical compositions may be varied so as to obtain an amount of the active ingredient which is effective to achieve the desired therapeutic response for a particular subject, composition, and mode of administration, without being toxic to the subject. The selected dosage level will depend upon a variety of factors including the activity of the particular compound, the route of administration, the time of administration, the rate of excretion or metabolism, the rate and extent of absorption, the duration of the treatment, other drugs, compounds and/or materials used in combination with the particular compound, the age, sex, weight, condition, general health and prior medical history of the subject being treated, and like factors well known in the medical arts. A physician or veterinarian having ordinary skill in the art can readily determine and prescribe the effective amount of the compound or pharmaceutical composition required. For example, the physician or veterinarian could start with doses at levels lower than that required in order to achieve the desired therapeutic effect and gradually increase the dosage until the desired effect is achieved.

In general, a suitable dose of a compound will be that amount of the compound which is the lowest safe and effective dose to produce a therapeutic effect. Such an effective dose will generally depend upon the factors described above. For example, intravenous doses of the compounds of the present invention for a subject may range from about 10 mg to about 1000 mg per meter² dosed twice per week, or between about 75 mg to 750 mg per meter² dosed twice per week, or 100 mg to 500 mg per meter² dosed twice per week.

General considerations in the manufacture of pharmaceutical compositions may be found, for example, in Remington: The Science and Practice of Pharmacy 21^{st} ed., Lippincott Williams & Wilkins, 2005.

Although the descriptions of pharmaceutical compositions provided herein are principally suitable for administration to humans, it will be understood by the skilled artisan that such pharmaceutical compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### EXEMPLIFICATION

The methods now being generally described, they will be more readily understood by reference to the following examples, which are included merely for purposes of illustration of certain aspects and embodiments, and are not intended to limit the methods.

Air-stable hydroquinone salts according to formulae I-V can be synthesized by using organic or inorganic acids. Suitable acids include, but are not limited to, HCl, HBr, H₂SO₄, methansulfonic acid, benzenesulfonic acid, p-toluenesulfonic acid, triflic acid, camphorsulfonic acid, naphthalene-1,5-disulfonic acid, ethan-1,2-disulfonic acid, cyclamic acid, thiocyanic acid, naphthalene-2-sulfonic acid, oxalic acid, and the like (see, e.g., Berge et al. “Pharmaceutical Salts”, J. Pharm. Sci. (1977) 66:1-19). Suitable acids have a pKa sufficient to protonate the aniline nitrogen. Thus, any acid with a pKa between about −10 and about 4, or between about −10 and about 3, or between about −10 and about 1, or between about −10 and about −3 may be used to generate the hydroquinone salt. Further, amino acids are represented in zwitterionic form (i.e., an internal salt) and can also be further protonated and exist as a salt. Exemplary synthetic routes are set forth herein and in U.S. Pat. Nos. 7,282,493 and 7,361,647 and 7,375,217, which are incorporated by reference in their entireties herein.

### Example 1

### Preparation of the Hydrochloride Salt of the Hydroquinone of 17-AAG

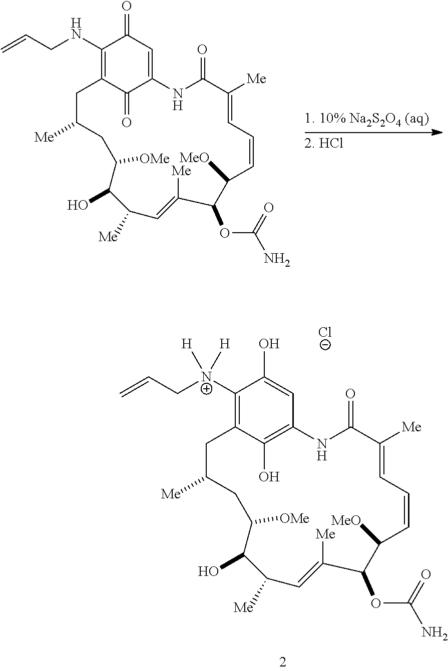

17-AAG (0.450 g, 0.768 mmol, 1.0 equiv) is dissolved in dichloromethane (50 mL) and stirred with a 10% aqueous solution of sodium hydrosulfite (50 mL). The solution is stirred for 30 minutes. The organic layer was collected, dried over Na₂SO₄, filtered and transferred to a round bottom flask. To this solution was added a solution of HCl in dioxane (4 N, 0.211 mL, 1.1 equiv.). The resulting mixture was allowed to stir under nitrogen for 30 minutes. A yellow solid slowly crashed out of solution. The yellow solid was purified by recrystallization form MeOH/EtOAc to yield 0.386 g of the hydroquinone HCl salt (2).

### Example 2

### Preparation of the Hydrobromide Salt of the Hydroquinone of 17-AAG

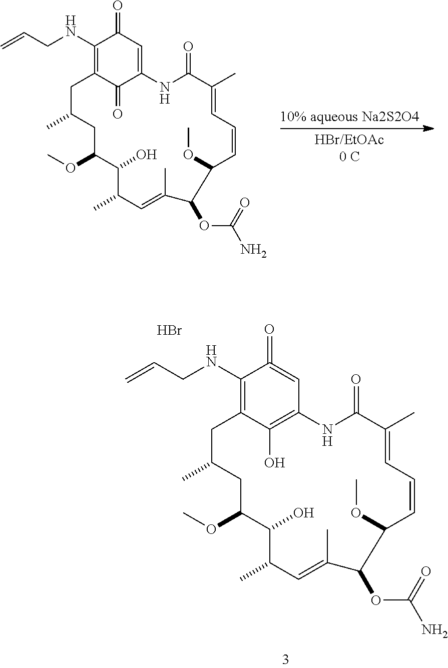

17-AAG (1.0 g, 1.71 mmol) in ethyl acetate (20 mL) was stirred vigorously with a freshly prepared solution of 10% aqueous sodium hydrosulfite (2 g in 20 mL water) for 30 minutes at ambient temperature. The color changed from dark purple to bright yellow, indicating a complete reaction. The layers were separated and the organic phase was dried with magnesium sulfate (1 g). The reaction solvent was collected and the drying agent was rinsed with ethyl acetate (1 mL). The combined filtrate was cooled to 0 C and acidified with 1.5 M hydrogen bromide in ethyl acetate until a precipitate formed. The resulting slurry was stirred for 30 minutes at ambient temperature. The solids were isolated by filtration, rinsed with ethyl acetate (1 mL) and dried at 40° C., 1 mm Hg, for 16 h to afford 0.352 g (31%) of 3 as a off-white solid.

### Example 3

### Preparation of the Sulfate Salt of the Hydroquinone of 17-AAG

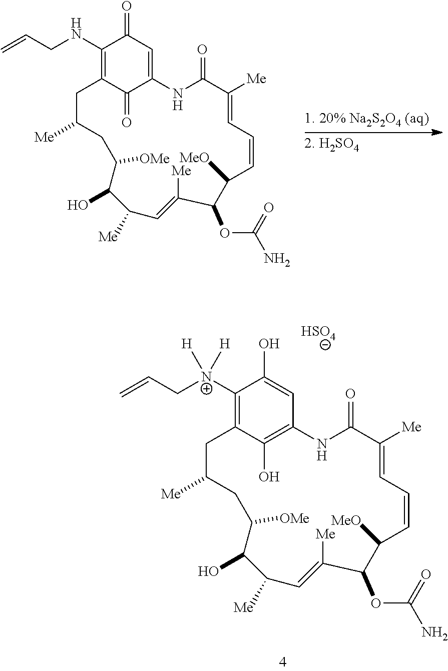

17-AAG (0.30 g, 0.5 mmol, 1.0 equiv) is dissolved in MTBE (3 mL) and stirred with a 20% aqueous solution of sodium hydrosulfite (2 mL). The solution is stirred for 60 minutes. The organic layer was collected, washed with brine, and transferred to a round bottom flask. This solution was cooled −5° C. and put under nitrogen. To this solution was added a solution of H₂SO₄ in denatured ethanol (0.50 mmol of H₂SO₄ in 0.5 mL of EtOH) dropwise. The resulting mixture was allowed to stir under nitrogen and warm to RT. The yellow slurry was stirred for an additional 30 minutes at RT and then was concentrated. MTBE (7 mL) was added and the suspension was filtered. The yellow solid that was collected was washed with MTBE and dried under reduced pressure to yield 0.30 g of the desired product 4.

### Example 4

### Preparation of the p-toluenesulfonate Salt of the Hydroquinone of 17-AAG

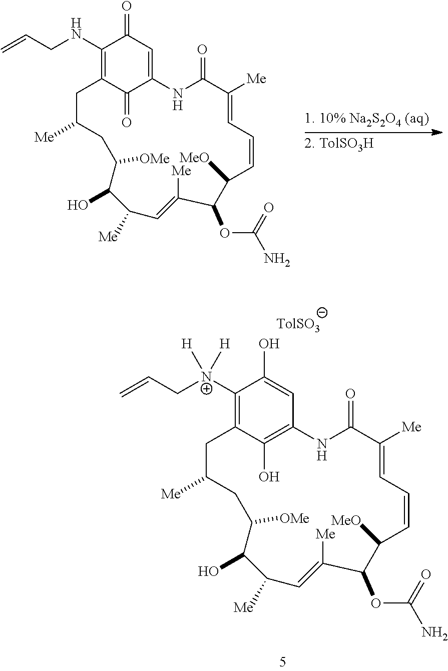

17-AAG (0.30 g, 0.5 mmol, 1.0 equiv) is dissolved in DCM (6 mL) and stirred with a 10% aqueous solution of sodium hydrosulfite (3.5 mL). The solution is stirred for 60 minutes. The organic layer was collected, washed with brine, and 1.2 mL (calc 0.1 mmol of hydroquinone) transferred to a round bottom flask. This solution was put under nitrogen. To this solution was added a solution of p-toluenesolfonic acid in denatured IPA (0.100 mmol of p-toluenesolfonic in 0.25 mL of IPA) dropwise. The resulting mixture was allowed to stir under nitrogen for 1 hour, at which point the mixture was concentrated and the crude mass was reslurried from EtOAc/MTBE. The solid was collected by filtration and dried under reduced pressure to yield 0.068 g of the desired product 5.

### Example 5

### Preparation of the d-camphorsulfonate Salt of the Hydroquinone of 17-AAG

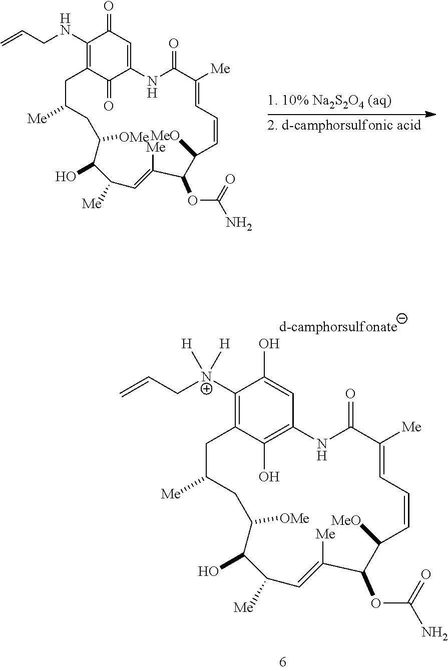

17-AAG (0.30 g, 0.5 mmol, 1.0 equiv) is dissolved in DCM (6 mL) and stirred with a 10% aqueous solution of sodium hydrosulfite (3.5 mL). The solution is stirred for 60 minutes. The organic layer was collected, washed with brine, and 1.2 mL (calc 0.1 mmol of hydroquinone) transferred to a round bottom flask. This solution was put under nitrogen. To this solution was added a solution of d-camphorsulonic acid in denatured IPA (0.100 mmol of d-camphorsulonic acid in 0.25 mL of IPA) dropwise. The resulting mixture was allowed to stir under nitrogen for 1 hour, at which point the mixture was concentrated and the crude mass was reslurried from EtOAc/MTBE. The solid was collected by filtration and dried under reduced pressure to yield 0.051 g of the desired product 6.

### Example 6

### Preparation of the Hydrogen Phosphate Salt of the Hydroquinone of 17-AAG

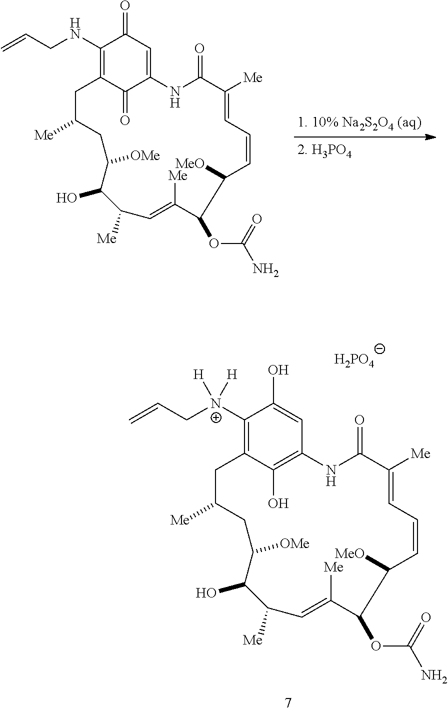

17-AAG (0.30 g, 0.5 mmol, 1.0 equiv) is dissolved in DCM (6 mL) and stirred with a 10% aqueous solution of sodium hydrosulfite (3.5 mL). The solution is stirred for 60 minutes. The organic layer was collected, washed with brine, and 1.2 mL (calc 0.1 mmol of hydroquinone) transferred to a round bottom flask. This solution was put under nitrogen. To this solution was added a solution of H₃PO₄ in denatured IPA (0.100 mmol of H₃PO₄ in 0.25 mL of IPA) dropwise. The resulting mixture was allowed to stir under nitrogen for 1 hour, at which point the mixture was concentrated and the crude mass was reslurried from EtOAc/MTBE. The solid was collected by filtration and dried under reduced pressure to yield 0.050 g of the desired product 7.

### Example 7

### Preparation of the Methylsulfonate Salt of the Hydroquinone of 17-AAG

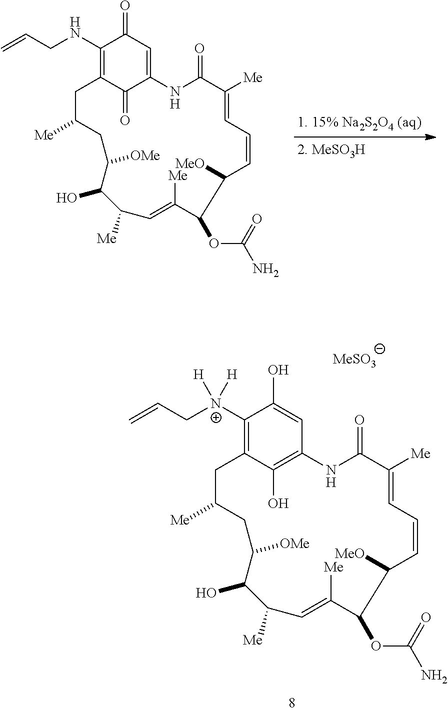

17-AAG (0.50 g, 0.8 mmol, 1.0 equiv) is dissolved in DCM (8 mL) and stirred with a 15% aqueous solution of sodium hydrosulfite (4 mL). The solution is stirred for 60 minutes. The organic layer was collected, washed with brine, and 2 mL (calc 0.2 mmol of hydroquinone) transferred to a round bottom flask. This solution was put under nitrogen. To this solution was added a solution of MeSO₃H in denatured IPA (0.200 mmol of MeSO₃H in 0.4 mL of IPA) dropwise. The resulting mixture was allowed to stir under nitrogen for 1 hour, at which point the mixture was concentrated and the crude mass was reslurried from EtOAc. The solid was collected by filtration and dried under reduced pressure to yield 0.112 g of the desired product 8.

### Example 8

### Preparation of the Benzenesulfonate Salt of the Hydroquinone of 17-AAG

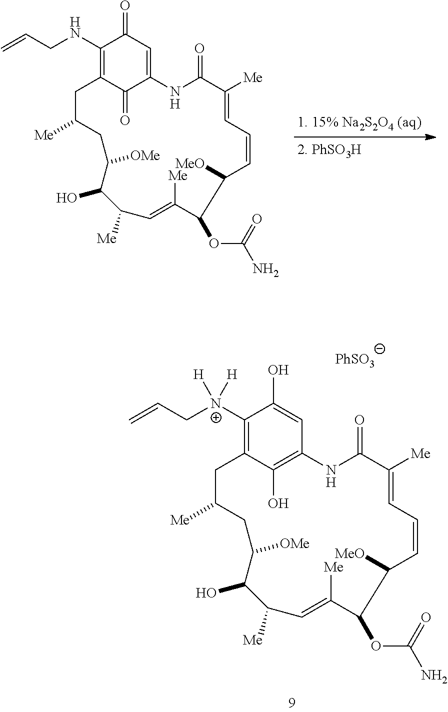

17-AAG (0.50 g, 0.8 mmol, 1.0 equiv) is dissolved in DCM (8 mL) and stirred with a 15% aqueous solution of sodium hydrosulfite (4 mL). The solution is stirred for 60 minutes. The organic layer was collected, washed with brine, and 2 mL (calc 0.2 mmol of hydroquinone) transferred to a round bottom flask. This solution was put under nitrogen. To this solution was added a solution of PhSO₃H in denatured IPA (0.200 mmol of PhSO₃H in 0.4 mL of IPA) dropwise. The resulting mixture was allowed to stir under nitrogen for 1 hour, at which point the mixture was concentrated and the crude mass was reslurried from EtOAc. The solid was collected by filtration and dried under reduced pressure to yield 0.118 g of the desired product 9.

### Example 9

### Preparation of Dimethylamino Acetate Co-Salt of the Hydroquinone of 17-AAG

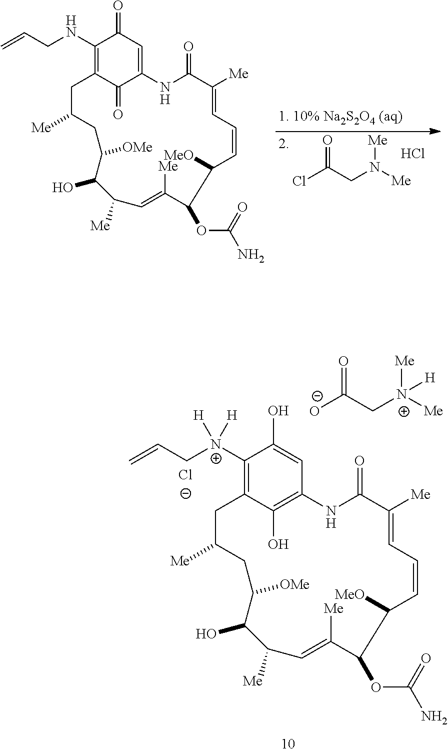

17-AAG (9.1 mg, 0.016 mmol, 1.0 equiv) was dissolved in 1.0 mL dichloromethane and stirred with a 10% aqueous solution of sodium hydrosulfite (1.0 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of dimethylaminoacetyl acid chloride hydrochloride (2.5 mg, 0.016 mmol, 1.0 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 10 as a white fluffy powder (7.1 mg, 0.011 mmol, 66% yield). The material was analyzed by ¹H NMR in D₂O and LC-MS.

### Example 10

### Preparation of α-Aminoisobutyrate Co-Salt of the Hydroquinone of 17-AAG

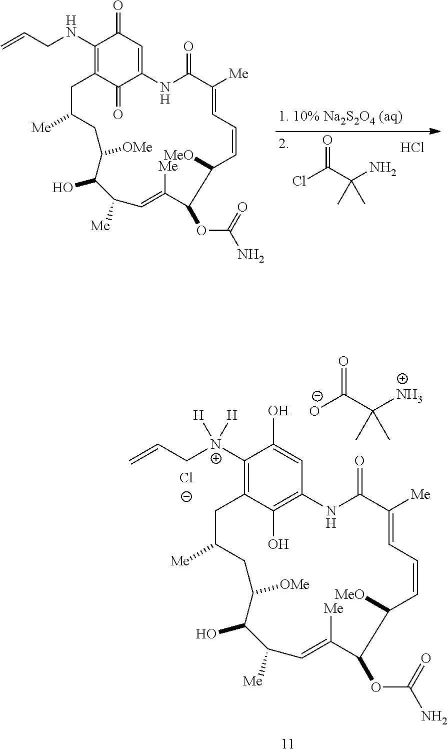

17-AAG (16.7 mg, 0.0285 mmol, 1.0 equiv) was dissolved in 1.5 mL dichloromethane and stirred with a 10% aqueous solution of sodium hydrosulfite (1.5 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of acid chloride hydrochloride (4.4 mg, 0.0314 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 11 as a white fluffy powder (15.1 mg, 0.0224 mmol, 79% yield). The material was analyzed by ¹H NMR in D₂O and LC-MS.

### Example 11

### Preparation of 13-Alanine Co-Salt of the Hydroquinone of 17-AAG

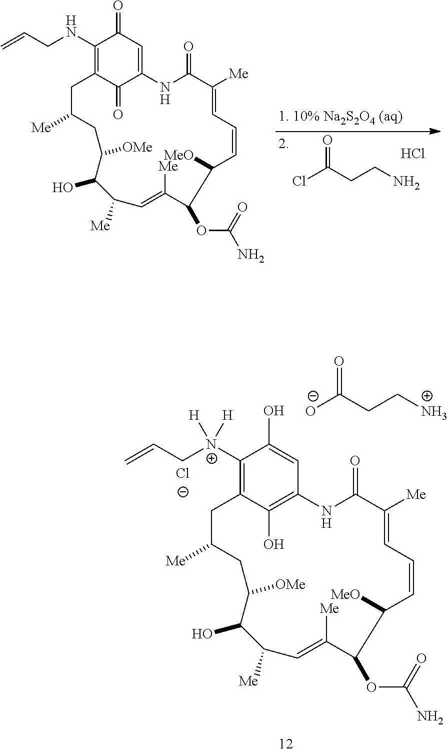

17-AAG (16.7 mg, 0.0285 mmol, 1.0 equiv) was dissolved in 1.5 mL dichloromethane and stirred with a 10% aqueous solution of sodium hydrosulfite (1.5 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of the acid chloride hydrochloride (4.52 mg, 0.0314 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 12 as a white fluffy powder (12 mg, 0.0237 mmol, 83% yield). The material was analyzed by ¹H NMR in D₂O and LC-MS.

### Example 12

### Preparation of N-Methyl Glycine Co-Salt of the Hydroquinone of 17-AAG

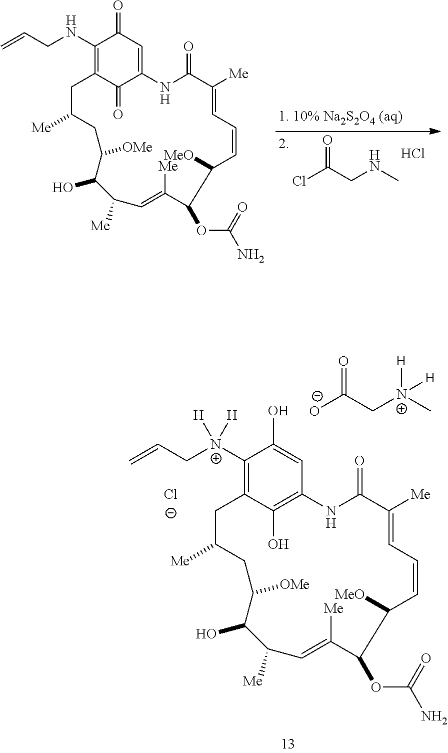

17-AAG (15.1 mg, 0.0258 mmol, 1.0 equiv) was dissolved in 1.5 mL dichloromethane and stirred with a 10% aqueous solution of sodium hydrosulfite (1.5 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of the acid chloride hydrochloride (3.7 mg, 0.0258 mmol, 1.0 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 13 as a white fluffy powder (15.4 mg, 0.0234 mmol, 91% yield). The material was analyzed by ¹H NMR in D₂O and LC-MS.

### Example 13

### Preparation of Piperidine Carboxylate Co-Salt of the Hydroquinone of 17-AAG

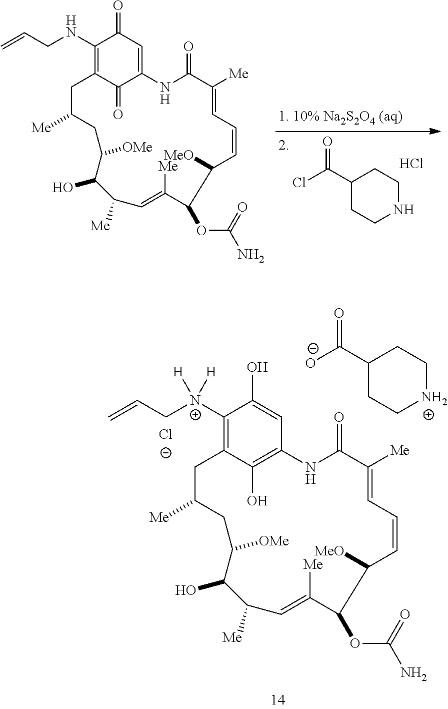

17-AAG (16 mg, 0.027 mmol, 1.0 equiv) was dissolved in 1.5 mL dichloromethane and stirred with a 10% aqueous solution of sodium hydrosulfite (1.5 mL). The deep purple solution turned yellow after 5 min and the mixture was stirred for an additional 25 min. The organic layer was removed via syringe and the aqueous solution was extracted with an additional 0.25 mL dichloromethane. The combined organic solutions were washed with brine (1.0 mL) and then added directly to a solution of the acid chloride hydrochloride (5.5 mg, 0.03 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 14 as a white fluffy powder (11.4 mg, 0.019 mmol, 60% yield). The material was analyzed by ¹H NMR in D₂O and LC-MS.

### Example 14

### Preparation of Glycine Co-Salt of the Hydroquinone of 17-AAG

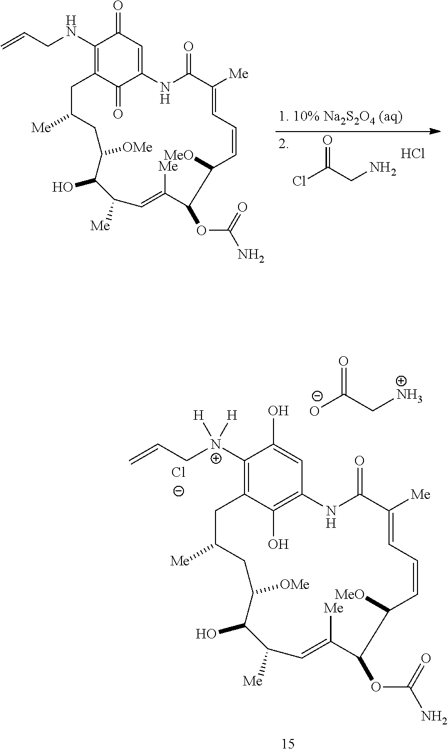

17-AAG (16.2 mg, 0.028 mmol, 1.0 equiv) was dissolved in 1.5 mL dichloromethane and stirred with a 10% aqueous solution of sodium hydrosulfite (1.5 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of the acid chloride hydrochloride (3.4 mg, 0.03 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 15 as a white fluffy powder (3.1 mg, 0.0051 mmol, 19% yield, 3:1 mixtures of phenol regioisomers). The material was analyzed by ¹H NMR in D₂O and LC-MS.

### Example 15

### Preparation of 2-Amino-2-ethyl-butyrate Co-Salt of the Hydroquinone of 17-AAG

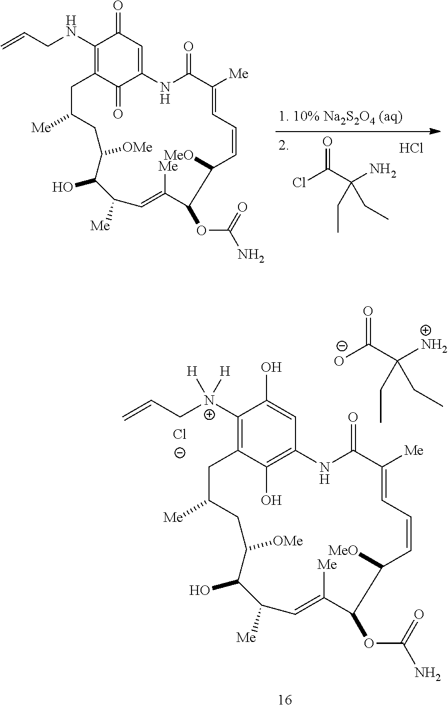

17-Allylaminogeldanamycin (1) (48 mg, 0.082 mmol, 1.0 equiv) was dissolved in 4.8 mL dichloromethane and stirred with a 10% aqueous solution of sodium hydrosulfite (4.8 mL). The deep purple solution turned yellow after 5 min and the mixture was stirred for an additional 25 min. The organic layer was removed via syringe and the aqueous solution was extracted with an additional 1 mL dichloromethane. The combined organic solutions were washed with brine (1.0 mL) and then added directly to a solution of the acid chloride hydrochloride (16.8 mg, 0.09 mmol, 1.1 equiv) in 1 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 16 as a white fluffy powder (24.7 mg, 0.034 mmol, 41% yield). The material was analyzed by ¹H NMR in D₂O and LC-MS.

### Example 16

### Preparation of 1-Amino-Cyclopropanecarboxylate Co-Salt of the Hydroquinone of 17-AAG

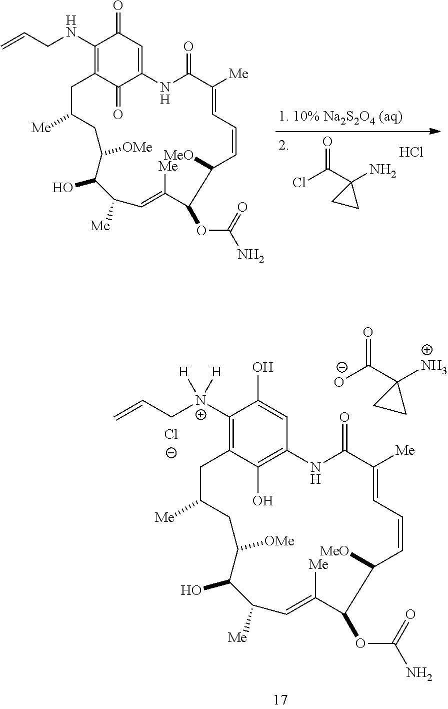

17-AAG (48 mg, 0.082 mmol, 1.0 equiv) was dissolved in 4.8 mL dichloromethane and stirred with a 10% aqueous solution of sodium hydrosulfite (4.8 mL). The deep purple solution turned yellow after 5 min and the mixture was stirred for an additional 25 min. The organic layer was removed via syringe and the aqueous solution was extracted with an additional 1 mL dichloromethane. The combined organic solutions were washed with brine (1.0 mL) and then added directly to a solution of the acid chloride hydrochloride (14.1 mg, 0.09 mmol, 1.1 equiv) in 1 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 17 as a white fluffy powder (36.2 mg, 0.051 mmol, 62% yield). The material was analyzed by ¹H NMR in D₂O and LC-MS.

### Example 17

### Preparation of 2-Methyl-2-(methylamino)propanoate Co-Salt of the Hydroquinone of 17-AAG

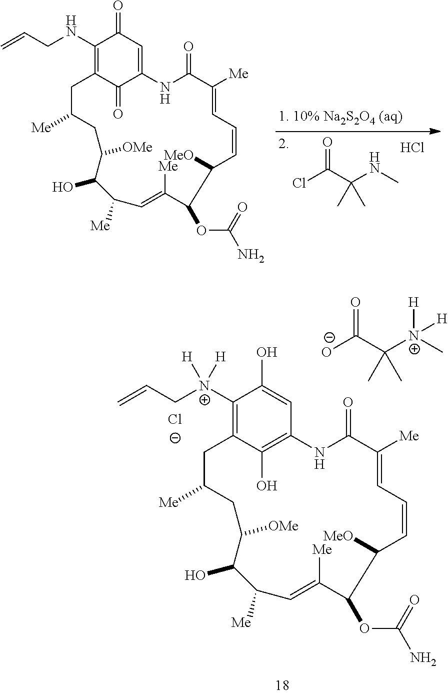

17-AAG (24 mg, 0.041 mmol, 1.0 equiv) was dissolved in 2.4 mL dichloromethane and stirred with a 10% aqueous solution of sodium hydrosulfite (2.4 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of the acid chloride hydrochloride (7.8 mg, 0.045 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 18 as a white fluffy powder (25.8 mg, 0.038 mmol, 92% yield). The material was analyzed by ¹H NMR in D₂O and LC-MS.

### Example 18

### Preparation of 1-Amino-cyclopentanecarboxylate Co-Salt of the Hydroquinone of 17-AAG

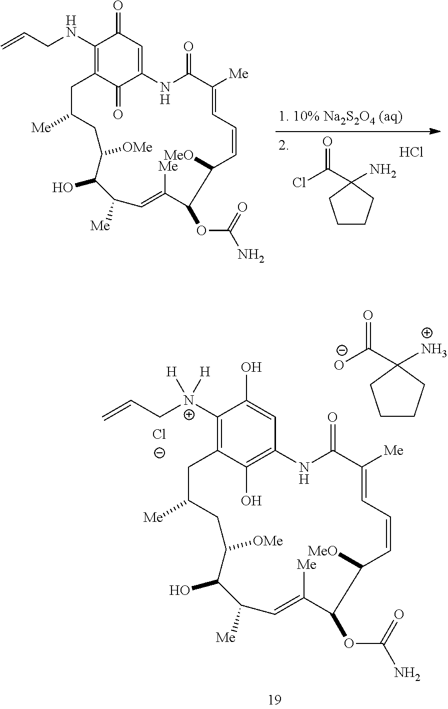

17-AAG (48 mg, 0.082 mmol, 1.0 equiv) was dissolved in 4.8 mL dichloromethane and stirred with a 10% aqueous solution of sodium hydrosulfite (4.8 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of the acid chloride hydrochloride (17 mg, 0.09 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 19 as a white fluffy powder (34.3 mg, 0.049 mmol, 60% yield). The material was analyzed by ¹H NMR in D₂O and LC-MS.

### Example 19

### Preparation of N-Methyl Piperidinecarboxylate Co-Salt of the Hydroquinone of 17-AAG

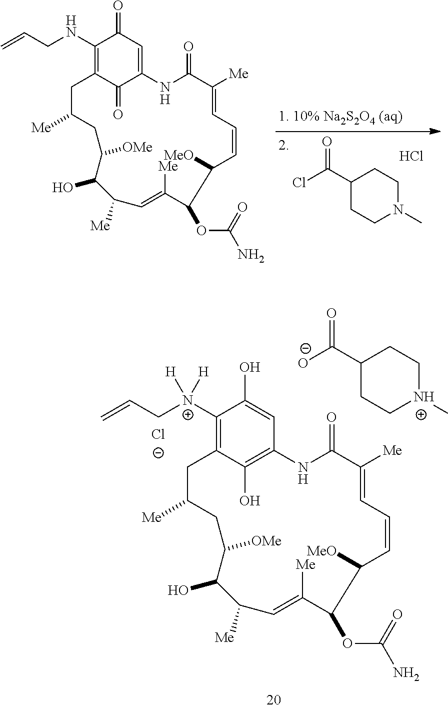

17-AAG (21.8 mg, 0.038 mmol, 1.0 equiv) was dissolved in 2 mL dichloromethane and stirred with a 10% aqueous solution of sodium hydrosulfite (2 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of the acid chloride hydrochloride (8.1 mg, 0.041 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 20 as a white fluffy powder (15.2 mg, 0.0213 mmol, 56% yield). The material was analyzed by ¹H NMR in D₂O and LC-MS.

### Example 20

### Preparation of N,N,N-Trimethylammonium Acetate Co-Salt of the Hydroquinone of 17-AAG

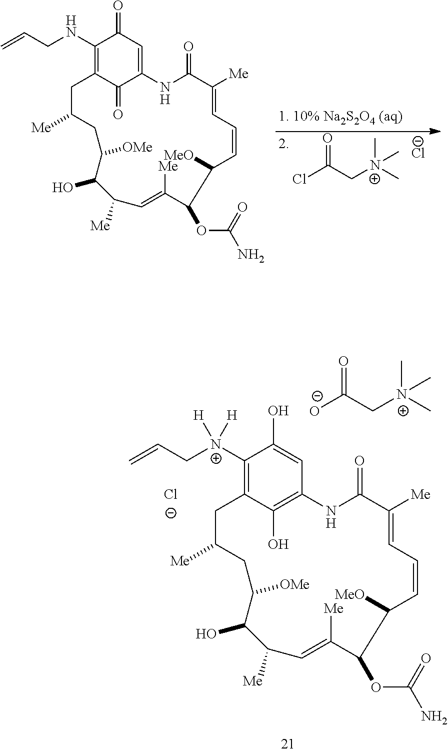

17-AAG (113 mg, 0.19 mmol, 1.0 equiv) was dissolved in 2 mL dichloromethane and stirred with a 10% aqueous solution of sodium hydrosulfite (2 mL). The deep purple solution turned yellow after 5 min and the mixture was stirred for an additional 25 min. The organic layer was removed via syringe and the aqueous solution was extracted with an additional 0.30 mL dichloromethane. The combined organic solutions were washed with brine (1.0 mL) and then added directly to a solution of the acid chloride hydrochloride (33 mg, 0.21 mmol, 1.1 equiv) in 0.20 mL dichloromethane. The reaction mixture was stirred for 2 h and poured into a separatory funnel with 3.0 mL water. The organic layer was extracted and then washed with additional 2.0 mL water. The combined aqueous layers were lyophilized to yield 21 as a white fluffy powder (78 mg, 0.11 mmol, 57% yield). The material was analyzed by ¹H NMR in CDCl₃/deuterated DMSO (6:1) and LC-MS.

### Example 21

### Preparation of a Liquid Formulation of Compound 2

For an 1 L preparation of formulation buffer, 9.6 g citric acid (USP), 8.8 g ascorbic acid (USP) and 1.0 g EDTA (Ethylenediamine-tetraacetic acid, disodium-calcium salt, dihydrate, USP), was added with a teflon-coated magnetic stir-bar to a 1 L volumetric flask. Sterile water for injection (USP) was added to 90-95% of the final volume of the flask. The solution was vigorously stirred to dissolve all solids. The pH of the buffer was adjusted to 3.1 using a NaOH solution. WFI was added to the final volume. The buffer was vacuum filtered through a 0.2 micron filter unit. Prior to use, the solution was sparged with nitrogen for 1-2 h. The formulation buffer was stored under nitrogen at 4° C. in a closed container.

A 10 mL volumetric flask was charged with solid compound 2 (500 mg) and purged with nitrogen. Formulation buffer (50 mM citrate, 50 mM ascorbate, 2.44 mM EDTA, pH 3.1) was sparged with nitrogen until dissolved oxygen content was <0.5 mg/L and chilled on ice. A portion of the buffer (approximately 5-7 mL) was added to the volumetric flask and vigorously shaken until all solid was dissolved. Buffer was then added to the 10 mL mark on the volumetric flask. The solution was kept cold on ice. A 10 mL syringe with syringe filter (Millipore, Durapore membrane, 0.2 micron) was used to filter the clear, slightly tan solution into a glass vial (USP Type I). The formulated compound 2 solution stored at 4° C. under a nitrogen headspace.

### Example 22

### Preparation of a Solid Formulation of Compound 2

52.50 g of sterile water was added to a 100 mL flask equipped with a magnetic stir bar. 6.305 g of citric acid monohydrate was added to the 100 mL flask and the resulting mixture was stirred until all of the citric acid dissolved into solution. 5.284 g of L-ascorbic acid was then added to the 100 mL flask and the solution was stirred until all of the ascorbic acid dissolved into solution.0.600 g of edetate calcium disodium was then added to the 100 mL flask and resulting mixture was stirred until all of the edetate calcium disodium had dissolved into solution. The pH of the solution was then adjusted to a pH of 3.1 by slowly adding a 5 M sodium hydroxide solution in water. The solution was then sparged with filtered (Millipak 20, 0.22 micron durapore) nitrogen for 2 hours. 52.04 g of the sparged solution was then cooled to 0° C. under nitrogen with stirring. 2.80 g of compound 2 was added and the resulting mixture was stirred until all of compound 2 was dissolved. This solution was sterile filtered using a 0.22 micron pore-size Durapore Millipak 200 filter at 0° C. The headspace of the receiving vessel was then flushed with filtered (Millipak 20, 0.22 micron durapore) nitrogen.

The receiving vessel was then placed in a lyophilizer, which had been pre-cooled to −40° C. The lyophilizer chamber was held at −40° C. for 3 hours at 1 atm. The pressure of the lyophilizer chamber was then ramped to 100 micron over one hour. Then the temperature of the chamber was ramped to −20° C. over 2 hour and the vacuum was held at 100 micron. The temperature of the chamber was then ramped to 0° C. over 2 hours and the vacuum was held at 100 micron. Then the temperature of the chamber was ramped to 0° C. over 2 hours and the vacuum was held at 100 micron. The temperature of the chamber was then ramped to +10° C. over 2 hours and the vacuum was held at 100 micron. Then the temperature of the chamber was ramped to +20° C. over 2 hours and the vacuum was held at 100 micron. The temperature of the chamber was then maintained at +20° C. for 48 hours and the vacuum was held at 100 micron. The chamber was then purged with nitrogen and a stopper was attached to the vessel containing the formulation. The formulation was stored at −20° C.

### Example 23

### Preparation of a Liquid Formulation of Compound 2

For a 1 L preparation of formulation buffer, 14.4 g citric acid, 30 g of ascorbic acid, 10 g of gamma-cyclodextrin (cyclooctaamylose) and 1.0 g EDTA was added with a teflon-coated magnetic stir-bar to a 1 L volumetric flask. Sterile water for injection was added to 90-95% of the final volume of the flask. The solution was vigorously stirred to dissolve all solids. The pH of the buffer was adjusted to 3.0 using a NaOH solution (NF grade). WFI was added to the final volume. The buffer was vacuum filtered through a 0.2 micron filter unit. Prior to use, the solution was sparged with nitrogen for 1-2 h. The formulation buffer was stored under nitrogen at 4° C. in a closed container.

The drug product was formulated at 4° C. by controlled dissolution of the solid compound 2 with pre-chilled nitrogen-sparged formulation buffer under a nitrogen headspace. Formulated compound 2 solution was stored at 4° C. under a nitrogen headspace.

### Example 24

### Preparation of a Liquid Formulation of Compound 2

For a 1 L preparation of formulation buffer, 9.6 g citric acid, 4.4 g of ascorbic acid, 10 mL of polysorbate-80 and 1.0 g EDTA (Ethylenediamine-tetraacetic acid, disodium-calcium salt, dihydrate) was added with a teflon-coated magnetic stir-bar to a 1 L volumetric flask. Sterile water for injection (WFI) was added to 90-95% of the final volume of the flask. The solution was vigorously stirred to dissolve all solids. The pH of the buffer was adjusted to 3.0 using a NaOH solution. WFI was added to the final volume. The buffer was vacuum filtered through a 0.2 micron filter unit. Prior to use, the solution was sparged with nitrogen for 1-2 h. The formulation buffer was stored under nitrogen at 4° C. in a closed container.

The drug product was formulated by controlled dissolution of the solid compound 2 with nitrogen-sparged formulation buffer. Formulated compound 2 solution stored at 4° C. under a nitrogen headspace.

### Example 25

### Administration of Compound 2 to a Subject with Dedifferentiated Metastatic Liposarcoma

A human subject with dedifferentiated metastatic liposarcoma was treated with compound 2 at 400 mg/m². The patient received two doses of compound 2 at 400 mg/m² during the first cycle of therapy. The patient received two doses at 300 mg/m² during the second cycle of therapy. The patient then received 225 mg/m² for seven cycles of therapy. Imaging assessment after the patient's first cycle of therapy revealed stable disease (tumor shrinkage of 28% using Response Evaluation Criteria in Solid Tumors (RECIST)). By the end of cycle 3 a partial response had been achieved (tumor shrinkage of 35%). The patient received a total of 9 cycles of compound 2 with maximal tumor shrinkage occurring after 7 cycles (54% tumor shrinkage).

### EQUIVALENTS

It is understood that the examples and embodiments described herein are for illustrative purposes only and that various modifications or changes in light thereof will be suggested to persons skilled in the art and are included within the spirit and purview of this application and scope of the appended claims.

1. A method of treating liposarcoma in a subject, the method comprising administering the subject a therapeutically effective amount of an Hsp90 inhibitor of formula I: 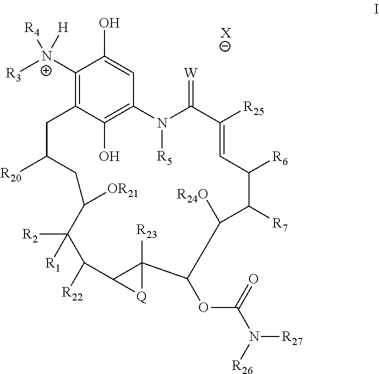
wherein independently for each occurrence:
W is oxygen or sulfur;
Q is oxygen, NR, NC(═O)R or a bond;
X⁻ is a conjugate base of a pharmaceutically acceptable acid;
R for each occurrence is independently selected from the group consisting of hydrogen, alkyl, carbocycyl, heterocycloalkyl, aralkyl, heteroaryl, and heteroaralkyl;
R₁ is alkoxyl, —OH, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;
R₃ and R₄ are each independently selected from the group consisting of hydrogen, alkyl, alkenyl, alkynyl, aryl, carbocycyl, heterocycloalkyl, aralkyl, heteroaryl, heteroaralkyl, and —[(C(R)₂)ₚ]—R₁₆; or R₃ taken together with R₄ represent a 4-8 membered optionally substituted heterocyclic ring;
R₅ is selected from the group consisting of H, alkyl, aralkyl, and a group having the formula Ia: 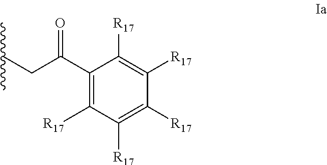
wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocycloalkyl;
R₆ and R₇ are both hydrogen; or R₆ and R₇ taken together form a bond;
R₈ is hydrogen, alkyl, alkenyl, alkynyl, aryl, carbocycyl, heterocycloalkyl, aralkyl, heteroaryl, heteroaralkyl, or —[(CR₂)ₚ]—R₁₆;
R₉ is alkyl, alkenyl, alkynyl, aryl, carbocycyl, heterocycloalkyl, aralkyl, heteroaryl, heteroaralkyl, or —[(CR₂)ₚ]—R₁₆;
R₁₀ and R₁₁ are each independently selected from the group consisting of hydrogen, alkyl, alkenyl, alkynyl, aryl, carbocycyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;
R₁₂ is alkyl, alkenyl, alkynyl, aryl, carbocycyl, heterocycloalkyl, aralkyl, heteroaryl, heteroaralkyl, or —[(C(R)₂)ₚ]—R₁₆;
R₁₃ and R₁₄ are each independently selected from the group consisting of hydrogen, alkyl, alkenyl, alkynyl, aryl, carbocycyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;
R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₈)(OR₁₉), and —P(O)(OR₁₈)(OR₁₉);
p is 1, 2, 3, 4, 5, or 6;
R₁₈ for each occurrence is independently selected from the group consisting of hydrogen, alkyl, aryl, carbocycyl, heterocycloalkyl, aralkyl, heteroaryl, and heteroaralkyl;
R₁₉ for each occurrence is independently selected from the group consisting of hydrogen, alkyl, aryl, carbocycyl, heterocycloalkyl, aralkyl, heteroaryl, and heteroaralkyl; or R₁₈ taken together with R₁₉ represent a 4-8 membered optionally substituted ring;
R₂₀, R₂₁, R₂₂, R₂₄, and R₂₅, for each occurrence are independently alkyl;
R₂₃ is alkyl, —CH₂OH, —CHO, —COOR₁₈, or —CH(OR₁₈)₂;
R₂₆ and R₂₇ for each occurrence are independently selected from the group consisting of hydrogen, alkyl, aryl, carbocycyl, heterocycloalkyl, aralkyl, heteroaryl, and heteroaralkyl;
provided that when R₁ is hydroxyl, R₂ is hydrogen, R₆ and R₇ taken together form a double bond, R₂₀ is methyl, R₂₁ is methyl, R₂₂ is methyl, R₂₃ is methyl, R₂₄ is methyl, R₂₅ is methyl, R₂₆ is hydrogen, R₂₇ is hydrogen, Q is a bond, and W is oxygen; R₃ and R₄ are not both hydrogen nor when taken together represent an unsubstituted azetidine; and
the absolute stereochemistry at a stereogenic center of formula 1 may be R or S or a mixture thereof and the stereochemistry of a double bond may be E or Z or a mixture thereof.

2. The method of claim 1, wherein the Hsp90 inhibitor is a compound of formula III: 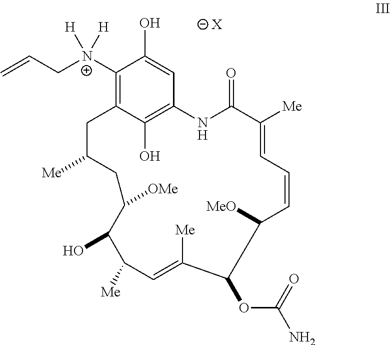
wherein X⁻ is the conjugate base of a pharmaceutically acceptable acid

3. The method of claim 2, wherein the pharmaceutically acceptable acid has a pKa of between about −10 and about 3.

4. The method of claim 2, wherein X⁻ is selected from the group consisting of chloride, bromide, iodide, H₂PO₄⁻, HSO₄⁻, methylsulfonate, benzenesulfonate, p-toluenesulfonate, trifluoromethylsulfonate, 10-camphorsulfonate, naphthalene-1-sulfonic acid-5-sulfonate, ethan-1-sulfonic acid-2-sulfonate, cyclamate, thiocyanate, naphthalene-2-sulfonate, and oxalate.

5. The method of claim 2, wherein X⁻ is chloride.

6. The method of claim 1, wherein the liposarcoma is selected from the group consisting of well differentiated liposarcoma, myxoid liposarcoma, round cell liposarcoma, pleomorphic liposarcoma, dedifferentiated liposarcoma, and mixed-type liposarcoma.

7. The method of claim 6, wherein the liposarcoma is characterized by over-expression of CDK4 protein.

8. The method of claim 6, wherein the liposarcoma is a dedifferentiated liposarcoma.

9. The method of claim 8, wherein the dedifferentiated liposarcoma characterized by over-expression of CDK4 protein.

10. The method of claim 6, wherein the liposarcoma is a well differentiated liposarcoma.

11. The method of claim 10, wherein the well differentiated liposarcoma is characterized by over-expression of CDK4 protein.

12. A method of treating liposarcoma in a subject, the method comprising administering the subject a therapeutically effective amount of an Hsp90 inhibitor of formula VI: 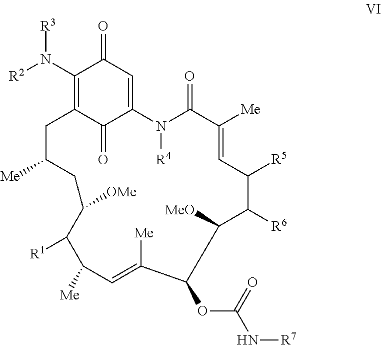
or a pharmaceutically acceptable salt thereof,
wherein:
R¹ is H, —OR⁸, —SR⁸—N(R⁸)(R⁹), —N(R⁸)C(O)R⁹, —N(R⁸)C(O)OR⁹, —N(R⁸)C(O)N(R⁸)(R⁹),
—OC(O)R⁸, —OC(O)OR⁸, —OS(O)₂R⁸, —OS(O)₂OR⁸, —OP(O)₂OR⁸, CN or ═O;
each of R² and R³ independently is H, alkyl, alkenyl, alkynyl, carbocycyl, cycloalkenyl, heterocycloalkyl, aryl, aralkyl, heteroaryl, heteroaralkyl, —C(═O)CH₃ or —[(C(R¹⁰)₂)ₚ]—R¹¹; or R² and R³ taken together with the nitrogen to which they are bonded represent a 3-8 membered optionally substituted heterocyclic ring which contains 1-3 heteroatoms selected from O, N, S, and P;
p independently for each occurrence is 0, 1, 2, 3, 4, 5, or 6;
R⁴ is H, alkyl, alkenyl, or aralkyl;
R⁵ and R⁶ are each H; or R⁵ and R⁶ taken together form a bond;
R⁷ is hydrogen alkyl, alkenyl, alkynyl, carbocycyl, cycloalkenyl, heterocycloaklyl, aryl, aralkyl, heteroaryl, heteroaralkyl, or —[(C(R¹⁰)₂)ₚ]—R¹¹;
each of R⁸ and R⁹ independently for each occurrence is H, alkyl, alkenyl, alkynyl, carbocycyl, cycloalkenyl, heterocycloaklyl, aryl, aralkyl, heteroaryl, heteroaralkyl, or —[(C(R¹⁰)₂)ₚ]—R¹¹; or R⁸ and R⁹ taken together represent a 3-8 membered optionally substituted heterocyclic ring which contains 1-3 heteroatoms selected from O, N, S, and P;
R¹⁰ for each occurrence independently is H, alkyl, alkenyl, alkynyl, carbocycyl, cycloalkenyl, heterocycloaklyl, aryl, aralkyl, heteroaryl, or heteroaralkyl; and
R¹¹ for each occurrence independently is H, carbocycyl, aryl, heteroaryl, heterocycloalkyl,
—OR⁸, —SR⁸, —N(R⁸)(R⁹), —N(R⁸)C(O)R⁹, —N(R⁸)C(O)OR⁹, —N(R⁸)C(O)N(R⁸)(R⁹), —OC(O)R⁸,
—OC(O)OR⁸, —OS(O)₂R⁸, —OS(O)₂OR⁸, —OP(O)₂OR⁸, —C(O)R⁸, —C(O)₂R⁸,
—C(O)N(R⁸)(R⁹), halide, or CN.

13. The method of claim 12, wherein the Hsp90 inhibitor is 17-AG.

14. The method of claim 13, wherein the 17-AG is substantially amorphous 17-AG.

15. The method of claim 12, wherein the liposarcoma is selected from the group consisting of well differentiated liposarcoma, myxoid liposarcoma, round cell liposarcoma, pleomorphic liposarcoma, dedifferentiated liposarcoma, and mixed-type liposarcoma.

16. The method of claim 15, wherein the liposarcoma is characterized by over-expression of CDK4 protein.

17. The method of claim 15, wherein the liposarcoma is a dedifferentiated liposarcoma.

18. The method of claim 17, wherein the dedifferentiated liposarcoma characterized by over-expression of CDK4 protein.

19. The method of claim 15, wherein the liposarcoma is a well differentiated liposarcoma.

20. The method of claim 19, wherein the well differentiated liposarcoma is characterized by over-expression of CDK4 protein.
